SEQUENCE LISTING (146 sequences)

SEQ ID NO: 1 - protein, 9 aa, Homo sapiens
  REGION 1..9: note = Description of sequence: RAS peptide
VVGADGVGK

SEQ ID NO: 2 - protein, 10 aa, Homo sapiens
  REGION 1..10: note = Description of sequence: RAS peptide
VVVGADGVGK

SEQ ID NO: 3 - protein, 10 aa, Homo sapiens
  REGION 1..10: note = Description of sequence: RAS peptide
VVVGAGGVGK

SEQ ID NO: 4 - protein, 7 aa, Homo sapiens
  REGION 1..7: note = Description of sequence: CDR 1 alpha-chain
TATGYPS

SEQ ID NO: 5 - protein, 7 aa, Homo sapiens
  REGION 1..7: note = Description of sequence: CDR2 alpha-chain
ATKADDK

SEQ ID NO: 6 - protein, 14 aa, Homo sapiens
  REGION 1..14: note = Description of sequence: CDR3 alpha-chain
CALSDRVGGARLMF

SEQ ID NO: 7 - protein, 5 aa, Homo sapiens
  REGION 1..5: note = Description of sequence: CDR 1 beta-chain
MGHDK

SEQ ID NO: 8 - protein, 6 aa, Homo sapiens
  REGION 1..6: note = Description of sequence: CDR2 beta-chain
SYGVNS

SEQ ID NO: 9 - protein, 13 aa, Homo sapiens
  REGION 1..13: note = Description of sequence: CDR3 beta-chain
CASSEGLYNEQFF

SEQ ID NO: 10 - protein, 132 aa, Homo sapiens
  REGION 1..132: note = Description of sequence: alpha-chain variable
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDRVGGARLMFGDGTQLVVKP

SEQ ID NO: 11 - protein, 131 aa, Homo sapiens
  REGION 1..131: note = Description of sequence: beta-chain variable
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGLYNEQFFGPGTRLTVL

SEQ ID NO: 12 - protein, 273 aa, Homo sapiens
  REGION 1..273: note = Description of sequence: Full alpha-chain
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDRVGGARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 310 aa, Homo sapiens
  REGION 1..310: note = Description of sequence: Full beta-chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGLYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 7 aa, Homo sapiens
  REGION 1..7: note = Description of sequence: CDR 1 alpha-chain
TSENNYY

SEQ ID NO: 15 - protein, 8 aa, Homo sapiens
  REGION 1..8: note = Description of sequence: CDR2 alpha-chain
QEAYKQQN

SEQ ID NO: 16 - protein, 16 aa, Homo sapiens
  REGION 1..16: note = Description of sequence: CDR3 alpha-chain
CAFMYPSQGGSEKLVF

SEQ ID NO: 17 - protein, 5 aa, Homo sapiens
  REGION 1..5: note = Description of sequence: CDR 1 beta-chain
SGHNT

SEQ ID NO: 18 - protein, 6 aa, Homo sapiens
  REGION 1..6: note = Description of sequence: CDR2 beta-chain
YYREEE

SEQ ID NO: 19 - protein, 15 aa, Homo sapiens
  REGION 1..15: note = Description of sequence: CDR3 beta-chain
CASSSPGFRSYGYTF

SEQ ID NO: 20 - protein, 137 aa, Homo sapiens
  REGION 1..137: note = Description of sequence: alpha-chain variable
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMYPSQGGSEKLVFGKGMKLTVNP

SEQ ID NO: 21 - protein, 133 aa, Homo sapiens
  REGION 1..133: note = Description of sequence: beta-chain variable
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSPGFRSYGYTFGSGTRLTVV

SEQ ID NO: 22 - protein, 278 aa, Homo sapiens
  REGION 1..278: note = Description of sequence: Full alpha-chain
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMYPSQGGSEKLVFGKGMKLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 310 aa, Homo sapiens
  REGION 1..310: note = Description of sequence: Full beta-chain
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSPGFRSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 24 - protein, 7 aa, Homo sapiens
  REGION 1..7: note = Description of sequence: CDR 1 alpha-chain
TSESDYY

SEQ ID NO: 25 - protein, 15 aa, Homo sapiens
  REGION 1..15: note = Description of sequence: CDR3 alpha-chain
CAYRSDGGATNKLIF

SEQ ID NO: 26 - protein, 5 aa, Homo sapiens
  REGION 1..5: note = Description of sequence: CDR 1 beta-chain
MNHEY

SEQ ID NO: 27 - protein, 6 aa, Homo sapiens
  REGION 1..6: note = Description of sequence: CDR2 beta-chain
SMNVEV

SEQ ID NO: 28 - protein, 16 aa, Homo sapiens
  REGION 1..16: note = Description of sequence: CDR3 beta-chain
CASSLGAGGYNSPLHF

SEQ ID NO: 29 - protein, 136 aa, Homo sapiens
  REGION 1..136: note = Description of sequence: alpha-chain variable
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDGGATNKLIFGTGTLLAVQP

SEQ ID NO: 30 - protein, 134 aa, Homo sapiens
  REGION 1..134: note = Description of sequence: beta-chain variable
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGAGGYNSPLHFGNGTRLTVT

SEQ ID NO: 31 - protein, 277 aa, Homo sapiens
  REGION 1..277: note = Description of sequence: Full alpha-chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 311 aa, Homo sapiens
  REGION 1..311: note = Description of sequence: Full beta-chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGAGGYNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 6 aa, Homo sapiens
  REGION 1..6: note = Description of sequence: CDR 1 alpha-chain
NSAFQY

SEQ ID NO: 34 - protein, 6 aa, Homo sapiens
  REGION 1..6: note = Description of sequence: CDR2 alpha-chain
TYSSGN

SEQ ID NO: 35 - protein, 17 aa, Homo sapiens
  REGION 1..17: note = Description of sequence: CDR3 alpha-chain
CAMGALNSGAGSYQLTF

SEQ ID NO: 36 - protein, 5 aa, Homo sapiens
  REGION 1..5: note = Description of sequence: CDR 1 beta-chain
SGHRS

SEQ ID NO: 37 - protein, 6 aa, Homo sapiens
  REGION 1..6: note = Description of sequence: CDR2 beta-chain
YFSETQ

SEQ ID NO: 38 - protein, 15 aa, Homo sapiens
  REGION 1..15: note = Description of sequence: CDR3 beta-chain
CASSLSSGTGTEAFF

SEQ ID NO: 39 - protein, 137 aa, Homo sapiens
  REGION 1..137: note = Description of sequence: alpha-chain variable
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGALNSGAGSYQLTFGKGTKLSVIP

SEQ ID NO: 40 - protein, 133 aa, Homo sapiens
  REGION 1..133: note = Description of sequence: beta-chain variable
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGTGTEAFFGQGTRLTVV

SEQ ID NO: 41 - protein, 278 aa, Homo sapiens
  REGION 1..278: note = Description of sequence: Full alpha-chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGALNSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 310 aa, Homo sapiens
  REGION 1..310: note = Description of sequence: Full beta-chain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGTGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 6 aa, Homo sapiens
  REGION 1..6: note = Description of sequence: CDR 1 alpha-chain
DSSSTY

SEQ ID NO: 44 - protein, 7 aa, Homo sapiens
  REGION 1..7: note = Description of sequence: CDR2 alpha-chain
IFSNMDM

SEQ ID NO: 45 - protein, 14 aa, Homo sapiens
  REGION 1..14: note = Description of sequence: CDR3 alpha-chain
CAERDAGNNRKLIW

SEQ ID NO: 46 - protein, 6 aa, Homo sapiens
  REGION 1..6: note = Description of sequence: CDR2 beta-chain
FQNEAQ

SEQ ID NO: 47 - protein, 13 aa, Homo sapiens
  REGION 1..13: note = Description of sequence: CDR3 beta-chain
CASSLEGGDTQYF

SEQ ID NO: 48 - protein, 133 aa, Homo sapiens
  REGION 1..133: note = Description of sequence: alpha-chain variable
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDAGNNRKLIWGLGTSLAVNP

SEQ ID NO: 49 - protein, 132 aa, Homo sapiens
  REGION 1..132: note = Description of sequence: beta-chain variable
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGDTQYFGPGTRLTVL

SEQ ID NO: 50 - protein, 274 aa, Homo sapiens
  REGION 1..274: note = Description of sequence: Full alpha-chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDAGNNRKLIWGLGTSLAVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 51 - protein, 311 aa, Homo sapiens
  REGION 1..311: note = Description of sequence: Full beta-chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 52 - protein, 27 aa, Homo sapiens
  REGION 1..27: note = Description of sequence: 2A peptide
RAKRSGSGATNFSLLKQAGDVEENPGP

SEQ ID NO: 53 - protein, 141 aa, Homo sapiens
  REGION 1..141: note = Description of sequence: Human alpha chain constant region
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 137 aa, Mus musculus
  REGION 1..137: note = Description of sequence: Mouse alpha chain constant region (cysteine-modification and LVL modification in transmembrane domain underlined)
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 55 - protein, 177 aa, Homo sapiens
  REGION 1..177: note = Description of sequence: Human beta chain constant region
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - protein, 173 aa, Mus musculus
  REGION 1..173: note = Description of sequence: Mouse beta chain constant region (cysteine-modification underlined)
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 57 - protein, 179 aa, Homo sapiens
  REGION 1..179: note = Description of sequence: Human beta chain constant region
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 58 - protein, 5 aa, Homo sapiens
  REGION 1..5: note = Description of sequence: CDR 1 beta-chain
SGHVS

SEQ ID NO: 59 - protein, 10 aa, Homo sapiens
  REGION 1..10: note = Description of sequence: GEM
AVFGAGGVGK

SEQ ID NO: 60 - protein, 10 aa, Homo sapiens
  REGION 1..10: note = Description of sequence: R-Ras
VVVGGGGVGK

SEQ ID NO: 61 - protein, 10 aa, Homo sapiens
  REGION 1..10: note = Description of sequence: ABCF1
STSPSDKVVK

SEQ ID NO: 62 - protein, 10 aa, Homo sapiens
  REGION 1..10: note = Description of sequence: BMS1
VVMGPPKVGK

SEQ ID NO: 63 - protein, 10 aa, Homo sapiens
  REGION 1..10: note = Description of sequence: CD166
NVFEAPTIVK

SEQ ID NO: 64 - protein, 10 aa, Homo sapiens
  REGION 1..10: note = Description of sequence: CFA47
MVFDSPTIGK

SEQ ID NO: 65 - protein, 10 aa, Homo sapiens
  REGION 1..10: note = Description of sequence: CK049
YSCPPPALVK

SEQ ID NO: 66 - protein, 10 aa, Homo sapiens
  REGION 1..10: note = Description of sequence: CL056
SSQSAPTTGK

SEQ ID NO: 67 - protein, 10 aa, Homo sapiens
  REGION 1..10: note = Description of sequence: CPNS1
AVMDSDTTGK

SEQ ID NO: 68 - protein, 10 aa, Homo sapiens
  REGION 1..10: note = Description of sequence: CPNS2
SVMDSDTTGK

SEQ ID NO: 69 - protein, 10 aa, Homo sapiens
  REGION 1..10: note = Description of sequence: FLNA
VTIDGPSKVK

SEQ ID NO: 70 - protein, 10 aa, Homo sapiens
  REGION 1..10: note = Description of sequence: GRIN1
GTAGPPSAVK

SEQ ID NO: 71 - protein, 10 aa, Homo sapiens
  REGION 1..10: note = Description of sequence: IGM
LTESGPALVK

SEQ ID NO: 72 - protein, 10 aa, Homo sapiens
  REGION 1..10: note = Description of sequence: KZF4
HSVSSPTVGK

SEQ ID NO: 73 - protein, 10 aa, Homo sapiens
  REGION 1..10: note = Description of sequence: IL17F
KTLHGPAMVK

SEQ ID NO: 74 - protein, 10 aa, Homo sapiens
  REGION 1..10: note = Description of sequence: K1671
TTKSGPALGK

SEQ ID NO: 75 - protein, 10 aa, Homo sapiens
  REGION 1..10: note = Description of sequence: KAD3
VIMGAPGSGK

SEQ ID NO: 76 - protein, 10 aa, Homo sapiens
  REGION 1..10: note = Description of sequence: LEGL
SVADSDAVVK

SEQ ID NO: 77 - protein, 10 aa, Homo sapiens
  REGION 1..10: note = Description of sequence: RL7A
KVAPAPAVVK

SEQ ID NO: 78 - protein, 10 aa, Homo sapiens
  REGION 1..10: note = Description of sequence: RSLAA
AVLGAPGVGK

SEQ ID NO: 79 - protein, 10 aa, Homo sapiens
  REGION 1..10: note = Description of sequence: SALL1
SATSPPGSVK

SEQ ID NO: 80 - protein, 10 aa, Homo sapiens
  REGION 1..10: note = Description of sequence: SEZ6
LSLEAPTVGK

SEQ ID NO: 81 - protein, 10 aa, Homo sapiens
  REGION 1..10: note = Description of sequence: SHAN3
TTVPSPASGK

SEQ ID NO: 82 - protein, 10 aa, Homo sapiens
  REGION 1..10: note = Description of sequence: GEM
AVFGAGGVGK

SEQ ID NO: 83 - protein, 10 aa, Homo sapiens
  REGION 1..10: note = Description of sequence: R-Ras
VVVGGGGVGK

SEQ ID NO: 84 - protein, 10 aa, Homo sapiens
  REGION 1..10: note = Description of sequence: CHADL
RQCGADKVGK

SEQ ID NO: 85 - protein, 10 aa, Homo sapiens
  REGION 1..10: note = Description of sequence: DAZP1
NNSGADEIGK

SEQ ID NO: 86 - protein, 10 aa, Homo sapiens
  REGION 1..10: note = Description of sequence: FMNL1
QEAGADTPGK

SEQ ID NO: 87 - protein, 10 aa, Homo sapiens
  REGION 1..10: note = Description of sequence: GTR14
QAHGADRSGK

SEQ ID NO: 88 - protein, 10 aa, Homo sapiens
  REGION 1..10: note = Description of sequence: GTR3
QAHGADRSGK

SEQ ID NO: 89 - protein, 10 aa, Homo sapiens
  REGION 1..10: note = Description of sequence: MICU1
YFFGADLKGK

SEQ ID NO: 90 - protein, 10 aa, Homo sapiens
  REGION 1..10: note = Description of sequence: ADA2C
GAGGADGQGA

SEQ ID NO: 91 - protein, 10 aa, Homo sapiens
  REGION 1..10: note = Description of sequence: CC88B
LRLGADGAGS

SEQ ID NO: 92 - protein, 10 aa, Homo sapiens
  REGION 1..10: note = Description of sequence: DYH8
KVAGADGKGI

SEQ ID NO: 93 - protein, 10 aa, Homo sapiens
  REGION 1..10: note = Description of sequence: EFMT2
MSSGADGGGG

SEQ ID NO: 94 - protein, 10 aa, Homo sapiens
  REGION 1..10: note = Description of sequence: FOXL2
AGAGADGYGY

SEQ ID NO: 95 - protein, 10 aa, Homo sapiens
  REGION 1..10: note = Description of sequence: HMCN2
IKQGADGSGT

SEQ ID NO: 96 - protein, 10 aa, Homo sapiens
  REGION 1..10: note = Description of sequence: MARF1
LKLGADGSGP

SEQ ID NO: 97 - protein, 10 aa, Homo sapiens
  REGION 1..10: note = Description of sequence: MED13
NNDGADGMGI

SEQ ID NO: 98 - protein, 10 aa, Homo sapiens
  REGION 1..10: note = Description of sequence: MYLK
GGVGADGGGS

SEQ ID NO: 99 - protein, 10 aa, Homo sapiens
  REGION 1..10: note = Description of sequence: NDF2
TEQGADGAGR

SEQ ID NO: 100 - protein, 10 aa, Homo sapiens
  REGION 1..10: note = Description of sequence: PBX3
GHEGADGDGR

SEQ ID NO: 101 - protein, 10 aa, Homo sapiens
  REGION 1..10: note = Description of sequence: PER1
PLEGADGGGD

SEQ ID NO: 102 - protein, 10 aa, Homo sapiens
  REGION 1..10: note = Description of sequence: ZN646
PEDGADGWGP

SEQ ID NO: 103 - protein, 10 aa, Homo sapiens
  REGION 1..10: note = Description of sequence: HIC1
GVPGPDGKGK

SEQ ID NO: 104 - protein, 10 aa, Homo sapiens
  REGION 1..10: note = Description of sequence: TBL3
LSSGSDGLVK

SEQ ID NO: 105 - protein, 10 aa, Homo sapiens
  REGION 1..10: note = Description of sequence: TUTLA
ISQGADGRGK

SEQ ID NO: 106 - protein, 10 aa, Homo sapiens
  REGION 1..10: note = Description of sequence: GEM
AVFGAGGVGK

SEQ ID NO: 107 - protein, 10 aa, Homo sapiens
  REGION 1..10: note = Description of sequence: R-Ras
VVVGGGGVGK

SEQ ID NO: 108 - protein, 10 aa, Homo sapiens
  REGION 1..10: note = Description of sequence: BY55
RDPGIDGVGE

SEQ ID NO: 109 - protein, 10 aa, Homo sapiens
  REGION 1..10: note = Description of sequence: CO6A3
GDDGRDGVGS

SEQ ID NO: 110 - protein, 10 aa, Homo sapiens
  REGION 1..10: note = Description of sequence: CO8A2
GPPGVDGVGV

SEQ ID NO: 111 - protein, 10 aa, Homo sapiens
  REGION 1..10: note = Description of sequence: COBA1
GPAGQDGVGG

SEQ ID NO: 112 - protein, 10 aa, Homo sapiens
  REGION 1..10: note = Description of sequence: COIA1
GDPGKDGVGQ

SEQ ID NO: 113 - protein, 10 aa, Homo sapiens
  REGION 1..10: note = Description of sequence: EFGM
EVKGKDGVGA

SEQ ID NO: 114 - protein, 10 aa, Homo sapiens
  REGION 1..10: note = Description of sequence: MEIS1
HYGGMDGVGI

SEQ ID NO: 115 - protein, 10 aa, Homo sapiens
  REGION 1..10: note = Description of sequence: MEIS2
HYGGMDGVGV

SEQ ID NO: 116 - protein, 10 aa, Homo sapiens
  REGION 1..10: note = Description of sequence: PUR2
LASGTDGVGT

SEQ ID NO: 117 - protein, 10 aa, Homo sapiens
  REGION 1..10: note = Description of sequence: USH1G
EDGGLDGVGA

SEQ ID NO: 118 - protein, 10 aa, Homo sapiens
  REGION 1..10: note = Description of sequence: ABCF1
CIVGPNGVGK

SEQ ID NO: 119 - protein, 10 aa, Homo sapiens
  REGION 1..10: note = Description of sequence: AGRIN
PVCGSDGVTY

SEQ ID NO: 120 - protein, 10 aa, Homo sapiens
  REGION 1..10: note = Description of sequence: CGAT2
RNVGANGIGY

SEQ ID NO: 121 - protein, 10 aa, Homo sapiens
  REGION 1..10: note = Description of sequence: DNHD1
TVLGPNGVGK

SEQ ID NO: 122 - protein, 10 aa, Homo sapiens
  REGION 1..10: note = Description of sequence: IBP7
PVCGSDGTTY

SEQ ID NO: 123 - protein, 10 aa, Homo sapiens
  REGION 1..10: note = Description of sequence: NLRP9
VLEGPDGIGK

SEQ ID NO: 124 - protein, 10 aa, Homo sapiens
  REGION 1..10: note = Description of sequence: PRA19
KLFISDGCGY

SEQ ID NO: 125 - protein, 10 aa, Homo sapiens
  REGION 1..10: note = Description of sequence: SMC5
MIVGANGTGK

SEQ ID NO: 126 - protein, 10 aa, Homo sapiens
  REGION 1..10: note = Description of sequence: SO3A1
PVCGADGITY

SEQ ID NO: 127 - protein, 10 aa, Homo sapiens
  REGION 1..10: note = Description of sequence: SO5A1
PVCGSDGITY

SEQ ID NO: 128 - protein, 10 aa, Homo sapiens
  REGION 1..10: note = Description of sequence: SOCS7
GSGGGDGTGK

SEQ ID NO: 129 - protein, 10 aa, Homo sapiens
  REGION 1..10: note = Description of sequence: SUCB2
CAIIANGITK

SEQ ID NO: 130 - protein, 10 aa, Homo sapiens
  REGION 1..10: note = Description of sequence: GEM
AVFGAGGVGK

SEQ ID NO: 131 - protein, 10 aa, Homo sapiens
  REGION 1..10: note = Description of sequence: R-Ras
VVVGGGGVGK

SEQ ID NO: 132 - protein, 10 aa, Homo sapiens
  REGION 1..10: note = Description of sequence: FRPD4
KSKLADGEGK

SEQ ID NO: 133 - protein, 10 aa, Homo sapiens
  REGION 1..10: note = Description of sequence: TAF6
GATTADGKGK

SEQ ID NO: 134 - protein, 10 aa, Homo sapiens
  REGION 1..10: note = Description of sequence: TUTLA
ISQGADGRGK

SEQ ID NO: 135 - protein, 10 aa, Homo sapiens
  REGION 1..10: note = Description of sequence: DCAF7
ASVGADGSVR

SEQ ID NO: 136 - protein, 10 aa, Homo sapiens
  REGION 1..10: note = Description of sequence: ELP2
VSAAADSAVR

SEQ ID NO: 137 - protein, 10 aa, Homo sapiens
  REGION 1..10: note = Description of sequence: FRPD1
KVAAADGPAR

SEQ ID NO: 138 - protein, 10 aa, Homo sapiens
  REGION 1..10: note = Description of sequence: INO80
SSLAPDSLVR

SEQ ID NO: 139 - protein, 10 aa, Homo sapiens
  REGION 1..10: note = Description of sequence: KI26A
PVAGPDGLSK

SEQ ID NO: 140 - protein, 10 aa, Homo sapiens
  REGION 1..10: note = Description of sequence: PRP4
ASCAADGSVK

SEQ ID NO: 141 - protein, 10 aa, Homo sapiens
  REGION 1..10: note = Description of sequence: GEM
AVFGAGGVGK

SEQ ID NO: 142 - protein, 10 aa, Homo sapiens
  REGION 1..10: note = Description of sequence: R-Ras
VVVGGGGVGK

SEQ ID NO: 143 - protein, 25 aa, Homo sapiens
  REGION 1..25: note = Description of sequence: HRAS
QHKLRKLNPPDESGPGCMSCKCVLS

SEQ ID NO: 144 - protein, 25 aa, Homo sapiens
  REGION 1..25: note = Description of sequence: NRAS
QYRMKKLNSSDDGTQCCMGLPCVVM

SEQ ID NO: 145 - protein, 25 aa, Homo sapiens
  REGION 1..25: note = Description of sequence: KRAS4A
QYRLKKISKEEKTPGCVKIKKCIIM

SEQ ID NO: 146 - protein, 24 aa, Homo sapiens
  REGION 1..24: note = Description of sequence: KRAS4B
KHKEKMSKDGKKKKKKSKTKCVIM

(19) United States
(12) Patent Application Publication
(10) Pub. No.: US 2024/0091361 A1
(43) Pub. Date: Mar. 21, 2024

(54) T CELL RECEPTORS TARGETING RAS MUTATIONS AND USES THEREOF

(71) Applicant: MEMORIAL SLOAN-KETTERING CANCER CENTER, New York, NY (US)

(72) Inventors: Christopher A. KLEBANOFF, New York, NY (US); Smita S. CHANDRAN, New York, NY (US)

(73) Assignee: MEMORIAL SLOAN-KETTERING CANCER CENTER, New York, NY (US)

(21) Appl. No.: 18/517,835

(22) Filed: Nov. 22, 2023

Related U.S. Application Data
(63) Continuation of application No. PCT/US22/30814, filed on May 25, 2022.
(60) Provisional application No. 63/192,783, filed on May 25, 2021.

(51) Int. Cl. A61K 39/00; C07K 14/725

(52) U.S. Cl. CPC A61K 39/464464; A61K 39/4611; A61K 39/4632; C07K 14/7051

(57) ABSTRACT

The presently disclosed subject matter provides novel T cell receptors (TCRs) that target a mutated RAS protooncogene. The presently disclosed subject matter further provides cells comprising such TCRs, and methods of using such cells for treating cancers associated with RAS.

### CROSS-REFERENCE TO RELATED APPLICATIONS

This application is a Continuation of International Patent Application No. PCT/US22/30814, filed May 25, 2022, which claims priority to U.S. Provisional Application No.: 63/192,783, filed May 25, 2021, the contents of each of which are incorporated by reference in their entireties, and to each of which priority is claimed.

### SEQUENCE LISTING

This application contains a Sequence Listing which has been electronically submitted and is hereby incorporated by reference in its entirety. Said XML copy, created on Nov. 21, 2023, is named 0927870108_ST26.xml and is 189,100 bytes in size.

### 1. INTRODUCTION

The presently disclosed subject matter provides novel T cell receptors (TCRs) that target mutated RAS proto-oncogenes. The presently disclosed subject matter further provides cells comprising such TCRs, and methods of using such cells for treating cancers associated with mutated RAS.

### 2. BACKGROUND OF THE INVENTION

Cell-based immunotherapy is a therapy with curative potential for treating cancers. Immunoresponsive cells (e.g., T cells) may be modified to target tumor antigens through the introduction of genetic material coding for TCRs specific to selected antigens. Targeted T cell therapy using specific TCRs has shown clinical success in treating diverse solid and hematologic malignancies.

Collectively, the RAS proteins are the most mutated family of oncoproteins in human cancer. Patients with oncogenic mutations encoding a RAS protein (e.g., KRAS, NRAS, and HRAS) typically respond poorly to standard therapies. Activating oncogenic RAS mutations are frequently observed at residue positions 12, 13 and 61 in cancer patients. Among these, G12 is the most frequently mutated residue (89%) and it most often mutates to aspartate (G12D), valine (G12V), or cysteine (G12C). Accordingly, there are needs for novel therapeutic strategies to identify TCRs targeting epitopes derived from mutated RAS proteins. Further, there is unmet need for developing strategies capable of inducing potent cancer eradication with minimal toxicity and immunogenicity.

### 3. SUMMARY OF THE INVENTION

The presently disclosed subject matter provides T cell receptors (TCRs) targeting a RAS peptide that comprises a mutation. In certain embodiments, the RAS peptide comprises a G12 mutation. In certain embodiments, the RAS peptide comprises a G12D mutation. In certain embodiments, the RAS peptide is a 9-mer or a 10-mer. In certain embodiments, the RAS peptide is a 10-mer. In certain embodiments, the RAS peptide comprises or consists of the amino acid sequence set forth in SEQ ID NO: 1 or SEQ ID NO: 2. In certain embodiments, the RAS peptide comprises or consists of the amino acid sequence set forth in SEQ ID NO: 2.

In certain embodiments, the RAS peptide is associated with an HLA class I complex. In certain embodiments, the HLA class I complex is selected from an HLA-A, an HLA-B, and an HLA-C. In certain embodiments, the HLA class I complex is an HLA-A. In certain embodiments, the HLA-A is an HLA-A*03 superfamily member. In certain embodiments, the HLA-A*03 superfamily is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

In certain embodiments, the TCR comprises an extracellular domain that binds to the RAS peptide, wherein the extracellular domain comprises an α chain and a β chain, wherein the α chain comprises an α chain variable region and α chain constant region, and the β chain comprises a β chain variable region and a β chain constant region.

In certain embodiments, the extracellular domain comprises an α chain variable region and a β chain variable region, wherein:
a) the α chain variable region comprises a CDR3 comprising the amino acid sequence set forth in SEQ ID NO: 6 or a conservative modification thereof, and the β chain variable region comprises a CDR3 comprising the amino acid sequence set forth in SEQ ID NO: 9 or a conservative modification thereof;
b) the α chain variable region comprises a CDR3 comprising the amino acid sequence set forth in SEQ ID NO: 16 or a conservative modification thereof, and the β chain variable region comprises a CDR3 comprising the amino acid sequence set forth in SEQ ID NO: 19 or a conservative modification thereof;
c) the α chain variable region comprises a CDR3 comprising the amino acid sequence set forth in SEQ ID NO: 25 or a conservative modification thereof, and the β chain variable region comprises a CDR3 comprising the amino acid sequence set forth in SEQ ID NO: 28 or a conservative modification thereof;
d) the α chain variable region comprises a CDR3 comprising the amino acid sequence set forth in SEQ ID NO: 35 or a conservative modification thereof, and the β chain variable region comprises a CDR3 comprising the amino acid sequence set forth in SEQ ID NO: 38 or a conservative modification thereof; or
e) the α chain variable region comprises a CDR3 comprising the amino acid sequence set forth in SEQ ID NO: 45 or a conservative modification thereof, and the β chain variable region comprises a CDR3 comprising the amino acid sequence set forth in SEQ ID NO: 46 or a conservative modification thereof.

In certain embodiments,
a) the α chain variable region comprises a CDR2 comprising the amino acid sequence set forth in SEQ ID NO: 5 or a conservative modification thereof, and the β chain variable region comprises a CDR2 comprising the amino acid sequence set forth in SEQ ID NO: 8 or a conservative modification thereof;
b) the α chain variable region comprises a CDR2 comprising the amino acid sequence set forth in SEQ ID NO: 15 or a conservative modification thereof, and the β chain variable region comprises a CDR2 comprising the amino acid sequence set forth in SEQ ID NO: 18 or a conservative modification thereof;
c) the α chain variable region comprises a CDR2 comprising the amino acid sequence set forth in SEQ ID NO: 15 or a conservative modification thereof, and the β chain variable region comprises a CDR2 comprising the amino acid sequence set forth in SEQ ID NO: 27 or a conservative modification thereof;
d) the α chain variable region comprises a CDR2 comprising the amino acid sequence set forth in SEQ ID NO: 34 or a conservative modification thereof, and the β chain variable region comprises a CDR2 comprising the amino acid sequence set forth in SEQ ID NO: 37 or a conservative modification thereof; or
e) the α chain variable region comprises a CDR2 comprising the amino acid sequence set forth in SEQ ID NO: 44 or a conservative modification thereof, and the β chain variable region comprises a CDR2 comprising the amino acid sequence set forth in SEQ ID NO: 46 or a conservative modification thereof.

In certain embodiments,
a) the α chain variable region comprises a CDR1 comprising the amino acid sequence set forth in SEQ ID NO: 41 or a conservative modification thereof, and the β chain variable region comprises a CDR1 comprising the amino acid sequence set forth in SEQ ID NO: 7 or a conservative modification thereof;
b) the α chain variable region comprises a CDR1 comprising the amino acid sequence set forth in SEQ ID NO: 14 or a conservative modification thereof, and the β chain variable region comprises a CDR1 comprising the amino acid sequence set forth in SEQ ID NO: 17 or a conservative modification thereof;
c) the α chain variable region comprises a CDR1 comprising the amino acid sequence set forth in SEQ ID NO: 24 or a conservative modification thereof, and the β chain variable region comprises a CDR1 comprising the amino acid sequence set forth in SEQ ID NO: 26 or a conservative modification thereof;
d) the α chain variable region comprises a CDR1 comprising the amino acid sequence set forth in SEQ ID NO: 33 or a conservative modification thereof, and the β chain variable region comprises a CDR1 comprising the amino acid sequence set forth in SEQ ID NO: 36 or a conservative modification thereof; or
e) the α chain variable region comprises a CDR1 comprising the amino acid sequence set forth in SEQ ID NO: 43 or a conservative modification thereof, and the β chain variable region comprises a CDR1 comprising the amino acid sequence set forth in SEQ ID NO: 58 or a conservative modification thereof.

In certain embodiments,
a) the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
b) the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
c) the α chain variable region comprises a CDR1 comprising the amino acid sequence set forth in SEQ ID NO: 24; a CDR2 comprising the amino acid sequence set forth in SEQ ID NO: 15; and a CDR3 comprising the amino acid sequence set forth in SEQ ID NO: 25;
d) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; or
e) the α chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45.

In certain embodiments,
a) the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
b) the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
c) the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28;
d) the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; or
e) the β chain variable region comprises a CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47.

In certain embodiments,
a) the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
b) the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 15, and a CDR3 comprising the amino acid sequence set forth in SEQ ID NO: 25; and the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28;
d) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; or
e) the α chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the β chain variable region comprises a CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47.

In certain embodiments, the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

In certain embodiments, the α chain variable region comprises an amino acid sequence that is at least about 80% homologous or identical to the amino acid sequence set forth in SEQ ID NO: 10, SEQ ID NO: 20, SEQ ID NO: 29, SEQ ID NO: 39, or SEQ ID NO: 48. In certain embodiments, the α chain variable region comprises the amino acid sequence set forth in SEQ ID NO: 10, SEQ ID NO: 20, SEQ ID NO: 29, SEQ ID NO: 39, or SEQ ID NO: 48. In certain embodiments, the α chain variable region comprises the amino acid sequence set forth in SEQ ID NO: 39.

In certain embodiments, the β chain variable region comprises an amino acid sequence that is at least about 80% homologous or identical to the amino acid sequence set forth in SEQ ID NO: 11, SEQ ID NO: 21, SEQ ID NO: 30, SEQ ID NO: 40, or SEQ ID NO: 49. In certain embodiments, the β chain variable region comprises the amino acid sequence set forth in SEQ ID NO: 11, SEQ ID NO: 21, SEQ ID NO: 30, SEQ ID NO: 40, or SEQ ID NO: 49. In certain embodiments, the β chain variable region comprises the amino acid sequence set forth in SEQ ID NO: 40.

In certain embodiments,
a) the α chain variable region comprises an amino acid sequence that is at least about 80% homologous or identical to the amino acid sequence set forth in SEQ ID NO: 10, and the β chain variable region comprises an amino acid sequence that is at least about 80% homologous or identical to the amino acid sequence set forth in SEQ ID NO: 11;
b) the α chain variable region comprises an amino acid sequence that is at least about 80% homologous or identical to the amino acid sequence set forth in SEQ ID NO: 20, and the β chain variable region comprises an amino acid sequence that is at least about 80% homologous or identical to the amino acid sequence set forth in SEQ ID NO: 21;
c) the α chain variable region comprises an amino acid sequence that is at least about 80% homologous or identical to the amino acid sequence set forth in SEQ ID NO: 29, and the β chain variable region comprises an amino acid sequence that is at least about 80% homologous or identical to the amino acid sequence set forth in SEQ ID NO: 30;
d) the α chain variable region comprises an amino acid sequence that is at least about 80% homologous or identical to the amino acid sequence set forth in SEQ ID NO: 39, and the β chain variable region comprises an amino acid sequence that is at least about 80% homologous or identical to the amino acid sequence set forth in SEQ ID NO: 40; or
e) the α chain variable region comprises an amino acid sequence that is at least about 80% homologous or identical to the amino acid sequence set forth in SEQ ID NO: 48, and the β chain variable region comprises an amino acid sequence that is at least about 80% homologous or identical to the amino acid sequence set forth in SEQ ID NO: 49.

In certain embodiments,
a) the α chain variable region comprises the amino acid sequence set forth in SEQ ID NO: 10, and the β chain variable region comprises the amino acid sequence set forth in SEQ ID NO: 11;
b) the α chain variable region comprises the amino acid sequence set forth in SEQ ID NO: 20, and the β chain variable region comprises the amino acid sequence set forth in SEQ ID NO: 21;
c) the α chain variable region comprises the amino acid sequence set forth in SEQ ID NO: 29, and the β chain variable region comprises the amino acid sequence set forth in SEQ ID NO: 30;
d) the α chain variable region comprises the amino acid sequence set forth in SEQ ID NO: 39, and the β chain variable region comprises the amino acid sequence set forth in SEQ ID NO: 40; or
e) the α chain variable region comprises the amino acid sequence set forth in SEQ ID NO: 48, and the β chain variable region comprises the amino acid sequence set forth in SEQ ID NO: 49.

In certain embodiments, the α chain variable region comprises the amino acid sequence set forth in SEQ ID NO: 39, and the β chain variable region comprises the amino acid sequence set forth in SEQ ID NO: 40.

In certain embodiments,
a) the α chain comprises the amino acid sequence set forth in SEQ ID NO: 12, and the β chain comprises the amino acid sequence set forth in SEQ ID NO: 13;
b) the α chain comprises the amino acid sequence set forth in SEQ ID NO: 22, and the β chain comprises the amino acid sequence set forth in SEQ ID NO: 23;
c) the α chain comprises the amino acid sequence set forth in SEQ ID NO: 31, and the β chain comprises the amino acid sequence set forth in SEQ ID NO: 32;
d) the α chain comprises the amino acid sequence set forth in SEQ ID NO: 41, and the β chain comprising the amino acid sequence set forth in SEQ ID NO: 42; or
e) the α chain comprises the amino acid sequence set forth in SEQ ID NO: 50, and the β chain comprises the amino acid sequence set forth in SEQ ID NO: 51.

In certain embodiments, the α chain comprises the amino acid sequence set forth in SEQ ID NO: 41, and the β chain comprises the amino acid sequence set forth in SEQ ID NO: 42.

In certain embodiments, the extracellular domain binds to the same RAS peptide as a reference TCR or a functional fragment thereof, wherein the reference TCR or functional fragment thereof comprises α chain variable region and a β chain variable region, wherein:
a) the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
b) the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 15, and a CDR3 comprising the amino acid sequence set forth in SEQ ID NO: 25; and the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28;
d) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; or
e) the α chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the β chain variable region comprises a CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47.

In certain embodiments, the TCR is recombinantly expressed, and/or expressed from a vector. In certain embodiments, the TCR does not bind to a RAS peptide comprising or consisting of the amino acid sequence set forth in SEQ ID NO: 3.

In certain embodiments, the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 53 or SEQ ID NO: 54. In certain embodiments, the α chain constant region comprises the amino acid sequence set forth in SEQ ID NO: 53 or SEQ ID NO: 54.

In certain embodiments, the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 55, SEQ ID NO: 56, or SEQ ID NO: 57. In certain embodiments, the β chain constant region comprises the amino acid sequence set forth in SEQ ID NO: 55, SEQ ID NO: 56, or SEQ ID NO: 57.

The presently disclosed subject matter provides nucleic acids encoding the TCRs disclosed herein. The presently disclosed subject matter further provides cells comprising the TCR disclosed herein or the nucleic acids disclosed herein. In certain embodiments, the cell is transduced with the TCR. In certain embodiments, the TCR is constitutively expressed on the surface of the cell. In certain embodiments, the cell is an immunoresponsive cell. In certain embodiments, the cell is selected from the group consisting of a T cell, and a pluripotent stem cell from which a lymphoid cell may be differentiated. In certain embodiments, the cell is a T cell. In certain embodiments, the T cell is selected from the group consisting of a cytotoxic T lymphocyte (CTL), a regulatory T cell, a γδ T cell, a Natural Killer-T cell (NK-T), a stem cell memory T cell (T_{SCM}), a central memory T cell (T_{CM}), and an effector memory T cell (T_{EM}). In certain embodiments, the T cell is a γδ T cell. In certain embodiments, the T cell is a NK-T cell. In certain embodiments, the TCR or nucleic acid is integrated at a locus within the genome of the cell (e.g., T cell). In certain embodiments, the locus is selected from a TRAC locus, a TRBC locus, a TRDC locus, and a TRGC locus. In certain embodiments, the locus is a TRAC locus or a TRBC locus.

The presently disclosed subject matter also provides compositions comprising the cells disclosed herein. In certain embodiments, the composition is a pharmaceutical composition further comprising a pharmaceutically acceptable carrier.

Furthermore, the presently disclosed subject matter provides vectors comprising the nucleic acids disclosed herein. In certain embodiments, the vector is a γ-retroviral vector.

Additionally, the presently disclosed subject matter provides methods for producing a cell that binds to a RAS peptide that comprises a G12 mutation. In certain embodiments, the method comprises introducing into the cell the nucleic acid or the vector disclosed herein.

Furthermore, the presently disclosed subject matter provides methods of treating and/or preventing a tumor associated with RAS in a subject. In certain embodiments, the method comprises administering to the subject the cells or the compositions disclosed herein. In certain embodiments, the tumor is associated with a RAS mutation. In certain embodiments, the RAS mutation is a G12D mutation.

In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, non-melanoma skin cancer, salivary gland cancer, melanoma, and multiple myeloma. In certain embodiments, the tumor is pancreatic cancer. In certain embodiments, the tumor is colorectal cancer. In certain embodiments, the subject is a human. In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

Furthermore, the presently disclosed subject matter provides uses of the cells or compositions disclosed herein for treating and/or preventing a tumor associated with RAS in a subject. In certain embodiments, the tumor is associated with a RAS mutation. In certain embodiments, the RAS mutation is a G12D mutation. In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma. In certain embodiments, the tumor is pancreatic cancer. In certain embodiments, the subject is a human. In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11

### 4. BRIEF DESCRIPTION OF THE FIGURES

The following Detailed Description, given by way of example but not intended to limit the invention to specific embodiments described, may be understood in conjunction with the accompanying drawings.

FIGS. 1A-1C depict a functional screen to elucidate the HLA-restricted immunopeptidome of endogenously processed and presented shared, or “public”, neoantigens (NeoAgs) resulting from mutant KRAS proteins. FIG. 1A shows a schematic overview of the HLA immune-precipitation (IP)/tandem mass spectrometry (MS/MS) screen using COS-7 as an artificial antigen presenting cell (aAPC). FIG. 1B shows a validation MS “mirror” plot for an eluted HLA-A*11:01-restricted KRAS(G12D) peptide from the surface of PANC1, a pancreatic cancer cell line that physiologically expresses HLA-A*11:01 and KRAS(G12D) (top panel). A synthetic peptide was run as a control (bottom panel). FIG. 1C shows a measurement of the relative stability of the neopeptide/HLA complex on the cell surface of TAP1/2-deficient T2 cells electroporated with in vitro transcribed RNA encoding HLA-A*11:01. X=preferred HLA anchor residue; X=location of hotspot mutation. SEQ ID NOs: 1-3 are represented.

FIG. 2 depicts a graphical comparison of the amino acid sequence homology and location of hotspot mutations in the RAS family of oncoproteins. *=location of hotspot mutations; vertical bar=site of sequence variation between RAS family members. Zoom area shows the sequence of the hypervariable region of all four RAS proteins (SEQ ID NOs: 143-146).

FIGS. 3A and 3B depict the discovery and varibale chain description of a panel of HLA-A*11:01-restricted mutant RAS-specific TCR gene sequences. FIG. 3A shows T cells derived from either a HLA-A*11:01⁺ healthy-donors (HD) or HLA-A*11:01⁺ patients with a history of a KRAS(G12D) cancer stimulated in vitro with autologous antigen presenting cells presenting KRAS(G12D). Individual cultures were screened for the presence of mutant RAS-specific T cells using a higher-order peptide/HLA-I reagent loaded with the mass-spec identified mutant 10mer epitope (SEQ ID NO: 2). Positive wells were labeled with barcoded-dextramers and subjected to single-cell V(D)J sequencing to retrieve the paired αβTCR gene sequences of mutant RAS-specific T cell clonotypes. FIG. 3B shows five unique mutant RAS-specific TCRs that were retrieved from a healthy-donor (n=1) or patient-derived samples (n=4). All five TCRs were composed of unique alpha and beta variable chain segments and CDR3 loop lengths.

FIGS. 4A and 4B depict the functional validation and measurement of co-receptor dependency of healthy donor (HD) and patient-derived TCR gene sequences specific for a RAS(G12D) public NeoAg. FIG. 4A shows FACS plots validating the functionality of five genetically distinct HD and patient-derived TCR gene sequences. Non-specific T cells were individually transduced with the indicated TCR. The frequnecy of intra-cellular TNFα production is displayed after gating on transduced T cells following co-culture with Cos7 target cells electroporated with the genes encoding HLA-A*11:01 and either WT KRAS or KRAS(G12D). FIG. 4B shows a summary bar graph (n=3 replicates per condition) displaying the frequency (±standard errror of the mean, SEM) of intracellular TNFα production in open-repertoire CD8⁺ (left) or CD4⁺ (right) T cells expressing individual RAS-specific TCRs after co-culutre with Cos7 target cells electroporated with the genes encoding HLA-A*11:01 and either WT KRAS or KRAS(G12D).

FIG. 5 depicts reactivity to different length minimal epitopes (10mer versus 9mer) by individual RAS(G12D)-specific TCR panel members.

FIGS. 6A and 6B depict the functional avidity of T cells transduced with RAS-specific TCRs. FIG. 6A shows intracellular TNFα production determined in CD8⁺ (left) or CD4⁺ (right) TC⁺ T cells. FIG. 6B shows EC50 values for each individual TCR in CD8⁺ or CD4⁺ T cells.

FIGS. 7A and 7B depict recognition of endogenous levels of KRAS(G12D) in a pancreatic tumor line by mutant RAS-specific TCR panel members. FIG. 7A shows open-repertoire T cells retrovirally transduced with an individual retrieved TCR gene sequence and cocultured with either the cholangiocarcinoma HuCCT1 cell line in the presence or absence of a pan-HLA class-I blocking antibody. FIG. 7B shows open-repertoire T cells retrovirally transduced with an individual retrieved TCR gene sequence and cocultured with either the pancreatic cancer PANC-1 cell line in the presence or absence of a pan HLA class-I blocking antibody.

FIGS. 8A and 8B depict tumor cytolysis of an HLA-A*11:01-expressing KRAS(G12D) tumor line (PANC-1) by RAS-specific TCR panel members. FIG. 8A shows tumor lysis curves for individual library members in the presence or absence of a pan-class I blocking antibody. FIG. 8B shows peak tumor lysis measured at 48 h post coculture.

FIGS. 9A and 9B depict cross-protection potential of RAS public neoantigen (NeoAg)-specific TCRs against alternative mutant RAS proteins. FIG. 9A shows representative FACS plots demonstrating cross-protective function of RAS(G12D)-specific TCR (TCR4). FIG. 9B shows summary bar graph (n=3 replicates per condition) of the frequency of intracellular TNFα producing TCR⁺CD8⁺ T cells in response to WT versus mutant RAS isoforms.

FIGS. 10A-10E depict heatmaps showing the levels of INF-γ production relative to each index amino acid. The native RAS mutated peptide sequence and position are listed at the top of each individual heatmap (SEQ ID NO: 2). The substituted amino acid is identified along the individual Y-axis rows. Index peptide at each position is identified in dotted squares. The relative influence of each amino acid at every position was used to determine TCR “preference” of that amino acid substitution at every position within the peptide. The TCR logo plots thus generated are shown above each individual TCR heatmap. FIG. 10A shows the heatmap of TCR1. FIG. 10B shows the heatmap of TCR2. FIG. 10C shows the heatmap of TCR3. FIG. 10D shows the heatmap of TCR4. FIG. 10E shows the heatmap of TCR5.

FIGS. 11A-11E depict cross-reactivity potential of RAS-specific TCRs. The level of IFN-γ was determined by ELISA. IFN-γ levels are shown in pg/mL on the y-axis; background threshold, as identified by the dotted line, was set at 50 pg/mL. FIG. 11A shows the level of IFN-γ produced by TCR1 incubated with the individual peptide described in Table 7. FIG. 11B shows the level of IFN-γ produced by TCR2 incubated with the individual peptide described in Table 8. FIG. 11C shows the level of IFN-γ produced by TCR3 incubated with the individual peptide described in Table 9. FIG. 11D shows the level of IFN-γ produced by TCR4 incubated with the individual peptide described in Table 10. FIG. 11E shows the level of IFN-γ produced by TCR5 incubated with the individual peptide described in Table 11.

### 5. DETAILED DESCRIPTION OF THE INVENTION

The presently disclosed subject matter provides TCRs, targeting RAS comprising a mutation, e.g., a G12D mutation. Furthermore, the presently disclosed subject matter provides cells (e.g., T cells) comprising the RAS-targeted TCRs, and methods of using such cells for treating tumors associated with RAS mutation(s).

For purposes of clarity of disclosure and not by way of limitation, the detailed description is divided into the following subsections:
5.1. Definitions;
5.2. RAS;
5.3. TCRs;
5.4. Cells
5.5. Nucleic Acids and Genetic Modifications of Cell;
5.6. Formulations and Administration; and
5.7. Methods of Treatments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and (β-chain. Generally, a TCR comprises three CDRs in the α-chain variable region and three CDRs in the (β-chain variable region. CDRs provide the majority of contact residues for the binding of the TCR to the antigen or epitope. CDRs regions can be delineated using the Kabat system (Kabat, E. A., et al. (1991) Sequences of Proteins of Immunological Interest, Fifth Edition, U.S. Department of Health and Human Services, NIH Publication No. 91-3242), the Chothia numbering system (Chothia et al., J Mol Biol. (1987) 196:901-17), the AbM numbering system (Abhinandan et al., Mol. Immunol. 2008, 45, 3832-3839), or the IMGT numbering system (accessible at http://www.imgt.org/IMGTScientificChart/Numbering/EVIGTIGVLsuperfamily.html, http://www.imgt.org/IMGTindex/numbering.php). In certain embodiments, the CDRs regions are delineated using the IMGT numbering system.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and ^{e-100} indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “disease” refers to any condition or disorder that damages or interferes with the normal function of a cell, tissue, or organ. Examples of diseases include neoplasm or pathogen infection of a cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a tumor), prevent or delay the recurrence of a tumor, or otherwise reduce the pathological consequences of the disease (e.g., a tumor). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses; and non-human primates such as apes and monkeys.

### 5.2. RAS

RAS is a family of oncoproteins encoding small GTPases involved in regulating cell growth, differentiation and survival of cells. In humans, the RAS family includes HRAS, NRAS, and KRAS. The KRAS gene has two splice variants, KRAS4A and KRAS4B. The expression of all isoforms is nearly ubiquitous, although they show quantitative and qualitative differences in expression depending on the tissue and/or developmental stage.

RAS proteins contain two domains: a G domain that binds guanosine nucleotides, and a C-terminal hypervariable region. The G domain is highly conserved between HRAS, NRAS, KRAS4A and KRAS4B and is responsible for binding and hydrolysis of guanine nucleotides. The hypervariable regions undergo differential post-translational modifications that in turn direct isoform-specific subcellular organization. RAS proteins act as binary molecular switches and cycle between an inactive GDP-bound and active GTP-bound state. Upon activation, RAS proteins recruit and activate proteins like c-Raf and PI3-kinase that result in cell proliferation, migration and protection from apoptosis.

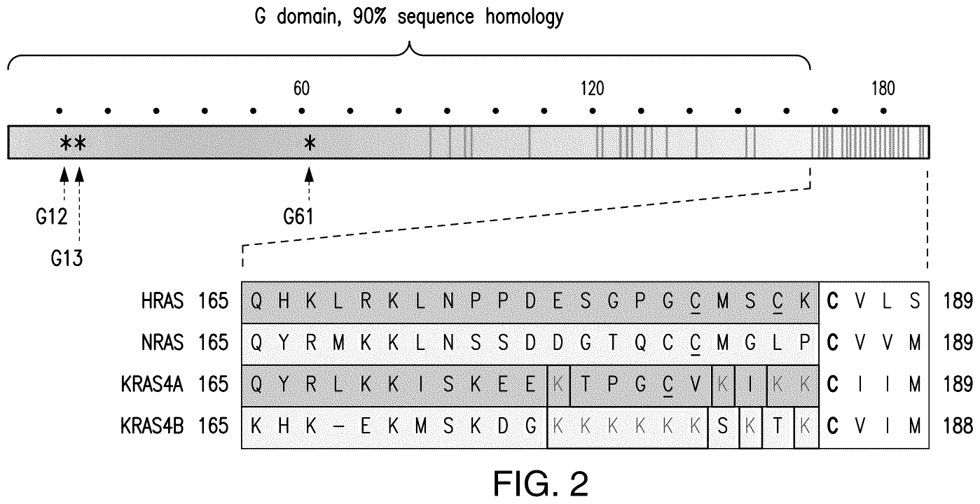

RAS mutations play a critical role in driving some of the most common and deadly carcinomas, including pancreatic, lung, and colorectal cancers, among numerous others. As illustrated in FIG. 2, the conserved G domain includes several locations for hotspot mutations including G12, G13, and Q61. The most frequent mutations of RAS genes occur at codon 12 (i.e., G12A/C/D/F/L/R/S/V), which accounts for 98% of RAS mutations. Across cancers, the most common RAS mutation is G12D, which is a single point mutation with a glycine-to-aspartate substitution at codon 12.

### 5.3. T-Cell Receptor (TCR)

A TCR is a disulfide-linked heterodimeric protein consisting of two variable chains expressed as part of a non-covalent complex with the invariant CD3 chain molecules (CD3δ, CD3ε, CD3γ, CD3ζ). A TCR is found on the surface of T cells, and is responsible for recognizing antigens bound to major histocompatibility complex (MEW) molecules. In certain embodiments, a TCR comprises an α chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247 ζ/ζ or ζ/ρ. When a TCR complex engages with its cognate peptide antigen/MHC (peptide/MHC), the T cell expressing the TCR complex is activated.

The presently disclosed subject matter provides recombinant TCRs. In certain embodiments, the recombinant TCR differs from any naturally occurring TCR by at least one amino acid residue. In certain embodiments, the recombinant TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the recombinant TCR is modified from a naturally occurring TCR by at least one amino acid residue. In certain embodiments, the recombinant TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the presently disclosed TCR targets or binds to a RAS peptide that comprises a mutation (“a mutant RAS peptide”). In certain embodiments, the mutation is a point mutation. In certain embodiments, the mutation is a G12 mutation. In certain embodiments, the RAS peptide comprises or consists of the amino acid sequence set forth in SEQ ID NO: 1. In certain embodiments, the RAS peptide comprises or consists of the amino acid sequence set forth in SEQ ID NO: 2. In certain embodiments, the presently disclosed TCR does not bind to a wildtype RAS. In certain embodiments, the presently disclosed TCR does not bind to a RAS peptide comprising or consisting of the amino acid sequence set forth in SEQ ID NO: 3. SEQ ID NOs: 1-3 are provided below.

[SEQ ID NO: 1]
VVGADGVGK
[SEQ ID NO: 2]
VVVGADGVGK
[SEQ ID NO: 3]
VVVGAGGVGK

In certain embodiments, the presently disclosed TCR targets or binds to KRAS comprising a RAS peptide comprising or consisting of the amino acid sequence set forth in SEQ ID NO: 1. In certain embodiments, the presently disclosed TCR targets or binds to KRAS comprising a RAS peptide comprising or consisting of the amino acid sequence set forth in SEQ ID NO: 2.

In certain embodiments, the presently disclosed TCR targets or binds to NRAS comprising a RAS peptide comprising or consisting of the amino acid sequence set forth in SEQ ID NO: 1. In certain embodiments, the presently disclosed TCR targets or binds to NRAS comprising a RAS peptide comprising or consisting of the amino acid sequence set forth in SEQ ID NO: 2.

In certain embodiments, the presently disclosed TCR targets or binds to HRAS comprising a RAS peptide comprising or consisting of the amino acid sequence set forth in SEQ ID NO: 1. In certain embodiments, the presently disclosed TCR targets or binds to HRAS comprising a RAS peptide comprising or consisting of the amino acid sequence set forth in SEQ ID NO: 2. In certain embodiments, the presently disclosed TCR targets or binds to a RAS peptide associated with an HLA class I complex, e.g., HLA-A, HLA-B and HLA-C.

In certain embodiments, the presently disclosed TCR targets or binds to a RAS peptide associated with an HLA-A*03 superfamily (e.g., in an HLA-A*03 superfamily dependent manner). In certain embodiments, the HLA*A03 superfamily members, include, but not limited to, alleles and sub-alleles in the HLA-A*03, HLA-A*11, HLA-A*31, HLA-A*33, HLA-A*66, HLA-A*68 and HLA-A*74. In certain embodiments, the presently disclosed TCR targets or binds to a RAS peptide associated with an HLA-A*11 molecule.

### 5.3.1. TCRs

### 5.3.1.1. Variable Regions

In certain embodiments, the extracellular domain of the TCR comprises an α chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOS: 4-6 are disclosed in Table 1. In certain embodiments, the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular domain of the TCR comprises a β chain variable region comprising a CDR1 comprising the amino acid sequence set forth in SEQ ID NO: 7 or a conservative modification thereof, a CDR2 comprising the amino acid sequence set forth in SEQ ID NO: 8 or a conservative modification thereof, and a CDR3 comprising the amino acid sequence set forth in SEQ ID NO: 9 or a conservative modification thereof. SEQ ID NOS: 7-9 are disclosed in Table 1. In certain embodiments, the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9.

In certain embodiments, the α chain variable region comprises a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; and the β chain variable region comprises a CDR1 comprising the amino acid sequence set forth in SEQ ID NO: 7 or a conservative modification thereof, a CDR2 comprising the amino acid sequence set forth in SEQ ID NO: 8 or a conservative modification thereof, and a CDR3 comprising the amino acid sequence set forth in SEQ ID NO: 9 or a conservative modification thereof In certain embodiments, the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 10. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 10. In certain embodiments, the α chain variable region comprises the amino acid sequence set forth in SEQ ID NO: 10. SEQ ID NO: 10 is provided in Table 1.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 11. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11. In certain embodiments, the β chain variable region comprises the amino acid sequence set forth in SEQ ID NO: 11. SEQ ID NO: 11 is provided in Table 1.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 10; and the β chain variable region comprises an amino acid sequence that is at least about 80% (e.g., at least about 85%, at least about 90%, or at least about 95%) homologous or identical to the amino acid sequence set forth in SEQ ID NO: 11. In certain embodiments, the α chain variable region comprises the amino acid sequence set forth in SEQ ID NO: 10; and the β chain variable region comprises the amino acid sequence set forth in SEQ ID NO: 11.

In certain embodiments, the extracellular domain of the TCR comprises an α chain that comprises an α chain variable region and an α chain constant region. In certain embodiments, the a chain comprises an amino acid sequence that is at least about 80% (e.g., at least about 85%, at least about 90%, or at least about 95%) homologous or identical to the amino acid sequence set forth in SEQ ID NO: 12. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 12. In certain embodiments, the α chain comprises the amino acid sequence set forth in SEQ ID NO: 12.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 13. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 13. In certain embodiments, the β chain comprises the amino acid sequence set forth in SEQ ID NO: 13.

In certain embodiments, the α chain comprises an amino acid sequence that is at least about 80% (e.g., at least about 85%, at least about 90%, or at least about 95%) homologous or identical to the amino acid sequence set forth in SEQ ID NO: 12; and the β chain comprises an amino acid sequence that is at least about 80% (e.g., at least about 85%, at least about 90%, or at least about 95%) homologous or identical to the amino acid sequence set forth in SEQ ID NO: 13. In certain embodiments, the α chain comprises the amino acid sequence set forth in SEQ ID NO: 12; and the β chain comprises the amino acid sequence set forth in SEQ ID NO: 13. In certain embodiments, the TCR is designated as “TCR 1”. In certain embodiments, the TCR1 binds to a RAS peptide comprising or consisting of the amino acid sequence set forth in SEQ ID NO: 2.

In certain embodiments, the CDRs sequences described above including Table 1 are delineated using the IMGT numbering system.

TABLE 1
(TCR1)

| CDRs | 1 | 2 | 3 |
|---|---|---|---|
| α-chain | TATGYPS | ATKADDK | CALSDRVGGARLMF |
| | [SEQ ID NO: 4] | [SEQ ID NO: 5] | [SEQ ID NO: 6] |
| β-chain | MGHDK | SYGVNS | CASSEGLYNEQFF |
| | [SEQ ID NO: 7] | [SEQ ID NO: 8] | [SEQ ID NO: 9] |

| | |
|---|---|
| α-chain | MNYSPGLVSLILLLLGRTRGDSVTQMEGPVTLSEEAFLTINCTYTATGYPSLFWYVQYPGEG |
| variable | LQLLLKATKADDKGSNKGFEATYRKETTSFHLEKGSVQVSDSAVYFCALSDRVGGARLMFGD |
| | GTQLVVKP[SEQ ID NO: 10] |
| β-chain | MTIRLLCYMGFYFLGAGLMEADIYQTPRYLVIGTGKKITLECSQTMGHDKMYWYQQDPGMEL |
| variable | HLIHYSYGVNSTEKGDLSSESTVSRIRTEHFPLTLESARPSHTSQYLCASSEGLYNEQFFGP |
| | GTRLTVL[SEQ ID NO: 11] |
| Full α- | MNYSPGLVSLILLLLGRTRGDSVTQMEGPVTLSEEAFLTINCTYTATGYPSLFWYVQYPGEG |
| chain | LQLLLKATKADDKGSNKGFEATYRKETTSFHLEKGSVQVSDSAVYFCALSDRVGGARLMFGD |
| | GTQLVVKPNIQNPDPAVYQLRDSKSSDKSVCLFTDFDSQTNVSQSKDSDVYITDKTVLDMRS |
| | MDFKSNSAVAWSNKSDFACANAFNNSIIPEDTFFPSPESSCDVKLVEKSFETDTNLNFQNLS |
| | VIGFRILLLKVAGENLLMTLRLWSS |
| | [SEQ ID NO: 12] |
| Full β- | MTIRLLCYMGFYFLGAGLMEADIYQTPRYLVIGTGKKITLECSQTMGHDKMYWYQQDPGMEL |
| chain | HLIHYSYGVNSTEKGDLSSESTVSRIRTEHFPLTLESARPSHTSQYLCASSEGLYNEQFFGP |
| | GTRLTVLEDLKNVFPPEVAVFEPSEAEISHTQKATLVCLATGFYPDHVELSWWVNGKEVHSG |
| | VSTDPQPLKEQPALNDSRYCLSSRLRVSATFWQNPRNHFRCQVQFYGLSENDEWTQDRAKPV |
| | TQIVSAEAWGRADCGFTSESYQQGVLSATILYEILLGKATLYAVLVSALVLMAMVKRKDSRG |
| | [SEQ ID NO: 13] |

In certain embodiments, the extracellular domain of the TCR comprises an α chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOS: 14-16 are disclosed in Table 2. In certain embodiments, the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular domain of the TCR comprises a β chain variable region comprising a CDR1 comprising the amino acid sequence set forth in SEQ ID NO: 17 or a conservative modification thereof, a CDR2 comprising the amino acid sequence set forth in SEQ ID NO: 18 or a conservative modification thereof, and a CDR3 comprising the amino acid sequence set forth in SEQ ID NO: 19 or a conservative modification thereof. SEQ ID NOS: 17-19 are disclosed in Table 2. In certain embodiments, the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19.

In certain embodiments, the α chain variable region comprises a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof; and the β chain variable region comprises a CDR1 comprising the amino acid sequence set forth in SEQ ID NO: 17 or a conservative modification thereof, a CDR2 comprising the amino acid sequence set forth in SEQ ID NO: 18 or a conservative modification thereof, and a CDR3 comprising the amino acid sequence set forth in SEQ ID NO: 19 or a conservative modification thereof. In certain embodiments, the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 20. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 20. In certain embodiments, the α chain variable region comprises the amino acid sequence set forth in SEQ ID NO: 20. SEQ ID NO: 20 is provided in Table 2.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 21. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21. In certain embodiments, the β chain variable region comprises the amino acid sequence set forth in SEQ ID NO: 21. SEQ ID NO: 21 is provided in Table 2. In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 20; and the β chain variable region comprises an amino acid sequence that is at least about 80% (e.g., at least about 85%, at least about 90%, or at least about 95%) homologous or identical to the amino acid sequence set forth in SEQ ID NO: 21. In certain embodiments, the α chain variable region comprises the amino acid sequence set forth in SEQ ID NO: 20; and the β chain variable region comprises the amino acid sequence set forth in SEQ ID NO: 21.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 22. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 22. In certain embodiments, the α chain comprises the amino acid sequence set forth in SEQ ID NO: 22.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 23. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23. In certain embodiments, the β chain comprises the amino acid sequence set forth in SEQ ID NO: 23.

In certain embodiments, the α chain comprises an amino acid sequence that is at least about 80% (e.g., at least about 85%, at least about 90%, or at least about 95%) homologous or identical to the amino acid sequence set forth in SEQ ID NO: 22; and the β chain comprises an amino acid sequence that is at least about 80% (e.g., at least about 85%, at least about 90%, or at least about 95%) homologous or identical to the amino acid sequence set forth in SEQ ID NO: 23. In certain embodiments, the α chain comprises the amino acid sequence set forth in SEQ ID NO: 22; and the β chain comprises the amino acid sequence set forth in SEQ ID NO: 23. In certain embodiments, the TCR is designated as “TCR 2”. In certain embodiments, the TCR2 binds to a RAS peptide comprising or consisting of the amino acid sequence set forth in SEQ ID NO: 2.

In certain embodiments, the CDRs sequences described above including Table 2 are delineated using the IMGT numbering system.

TABLE 2
(TCR2)

| CDRs | 1 | 2 | 3 |
|---|---|---|---|
| α-chain | TSENNYY [SEQ ID | QEAYKQQN [SEQ ID | CAFMYPSQGGSEKLVF [SEQ ID |
| | NO: 14] | NO: 15] | NO: 16] |
| β-chain | SGHNT [SEQ ID | YYREEE [SEQ ID | CASSSPGFRSYGYTF [SEQ ID |
| | NO: 17] | NO: 18] | NO: 19] |

| | |
|---|---|
| α-chain | MTRVSLLWAVVVSTCLESGMAQTVTQSQPEMSVQEAETVTLSCTYDTSENNYYLFWYKQPPS |
| variable | RQMILVIRQEAYKQQNATENRFSVNFQKAAKSFSLKISDSQLGDTAMYFCAFMYPSQGGSEK |
| | LVFGKGMKLTVNP [SEQ ID NO: 20] |
| β-chain | MGPGLLCWVLLCLLGAGSVETGVTQSPTHLIKTRGQQVTLRCSSQSGHNTVSWYQQALGQGP |
| variable | QFIFQYYREEENGRGNFPPRESGLQFPNYSSELNVNALELDDSALYLCASSSPGFRSYGYTF |
| | GSGTRLTVV [SEQ ID NO: 21] |
| Full α- | MTRVSLLWAVVVSTCLESGMAQTVTQSQPEMSVQEAETVTLSCTYDTSENNYYLFWYKQPPS |
| chain | RQMILVIRQEAYKQQNATENRFSVNFQKAAKSFSLKISDSQLGDTAMYFCAFMYPSQGGSEK |
| | LVFGKGMKLTVNPNIQNPDPAVYQLRDSKSSDKSVCLFTDFDSQTNVSQSKDSDVYITDKTV |
| | LDMRSMDFKSNSAVAWSNKSDFACANAFNNSIIPEDTFFPSPESSCDVKLVEKSFETDTNLN |
| | FQNLSVIGFRILLLKVAGENLLMTLRLWSS [SEQ ID NO: 22] |
| Full β- | MGPGLLCWVLLCLLGAGSVETGVTQSPTHLIKTRGQQVTLRCSSQSGHNTVSWYQQALGQGP |
| chain | QFIFQYYREEENGRGNFPPRESGLQFPNYSSELNVNALELDDSALYLCASSSPGFRSYGYTF |
| | GSGTRLTVVEDLNKVFPPEVAVFEPSEAEISHTQKATLVCLATGFFPDHVELSWWVNGKEVH |
| | SGVSTDPQPLKEQPALNDSRYCLSSRLRVSATFWQNPRNHFRCQVQFYGLSENDEWTQDRAK |
| | PVTQIVSAEAWGRADCGFTSVSYQQGVLSATILYEILLGKATLYAVLVSALVLMAMVKRKDF |
| | [SEQ ID NO: 23] |

In certain embodiments, the extracellular domain of the TCR comprises an α chain variable region comprising a CDR1 comprising the amino acid sequence set forth in SEQ ID NO: 24 or a conservative modification thereof, a CDR2 comprising the amino acid sequence set forth in SEQ ID NO: 15 or a conservative modification thereof, and a CDR3 comprising the amino acid sequence set forth in SEQ ID NO: 25 or a conservative modification thereof. SEQ ID NOS: 15, 24, and 25 are disclosed in Table 3. In certain embodiments, the α chain variable region comprises a CDR1 comprising the amino acid sequence set forth in SEQ ID NO: 24, a CDR2 comprising the amino acid sequence set forth in SEQ ID NO: 15, and a CDR3 comprising the amino acid sequence set forth in SEQ ID NO: 25.

In certain embodiments, the extracellular domain of the TCR comprises a β chain variable region comprising a CDR1 comprising the amino acid sequence set forth in SEQ ID NO: 26 or a conservative modification thereof, a CDR2 comprising the amino acid sequence set forth in SEQ ID NO: 27 or a conservative modification thereof, and a CDR3 comprising the amino acid sequence set forth in SEQ ID NO: 28 or a conservative modification thereof. SEQ ID NOS: 26-28 are disclosed in Table 3. In certain embodiments, the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28.

In certain embodiments, the α chain variable region comprises a CDR1 comprising the amino acid sequence set forth in SEQ ID NO: 24 or a conservative modification thereof, a CDR2 comprising the amino acid sequence set forth in SEQ ID NO: 15 or a conservative modification thereof, and a CDR3 comprising the amino acid sequence set forth in SEQ ID NO: 25 or a conservative modification thereof; and the β chain variable region comprises a CDR1 comprising the amino acid sequence set forth in SEQ ID NO: 26 or a conservative modification thereof, a CDR2 comprising the amino acid sequence set forth in SEQ ID NO: 27 or a conservative modification thereof, and a CDR3 comprising the amino acid sequence set forth in SEQ ID NO: 28 or a conservative modification thereof. In certain embodiments, the α chain variable region comprises a CDR1 comprising the amino acid sequence set forth in SEQ ID NO: 24, a CDR2 comprising the amino acid sequence set forth in SEQ ID NO: 15, and a CDR3 comprising the amino acid sequence set forth in SEQ ID NO: 25; and the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 29. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 29. In certain embodiments, the α chain variable region comprises the amino acid sequence set forth in SEQ ID NO: 29. SEQ ID NO: 29 is provided in Table 3.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 30. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 30. In certain embodiments, the β chain variable region comprises the amino acid sequence set forth in SEQ ID NO: 30. SEQ ID NO: 30 is provided in Table 3.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 29; and the β chain variable region comprises an amino acid sequence that is at least about 80% (e.g., at least about 85%, at least about 90%, or at least about 95%) homologous or identical to the amino acid sequence set forth in SEQ ID NO: 30. In certain embodiments, the α chain variable region comprises the amino acid sequence set forth in SEQ ID NO: 29; and the β chain variable region comprises the amino acid sequence set forth in SEQ ID NO: 30.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 31. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 31. In certain embodiments, the α chain comprises the amino acid sequence set forth in SEQ ID NO: 31.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 32. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 32. In certain embodiments, the β chain comprises the amino acid sequence set forth in SEQ ID NO: 32.

In certain embodiments, the α chain comprises an amino acid sequence that is at least about 80% (e.g., at least about 85%, at least about 90%, or at least about 95%) homologous or identical to the amino acid sequence set forth in SEQ ID NO: 31; and the β chain comprises an amino acid sequence that is at least about 80% (e.g., at least about 85%, at least about 90%, or at least about 95%) homologous or identical to the amino acid sequence set forth in SEQ ID NO: 32. In certain embodiments, the α chain comprises the amino acid sequence set forth in SEQ ID NO: 31; and the β chain comprises the amino acid sequence set forth in SEQ ID NO: 32. In certain embodiments, the TCR is designated as “TCR 3”. In certain embodiments, the TCR3 binds to a RAS peptide comprising or consisting of the amino acid sequence set forth in SEQ ID NO: 2.

In certain embodiments, the CDRs sequences described above including Table 3 are delineated using the IMGT numbering system.

TABLE 3
(TCR3)

| CDRs | 1 | 2 | 3 |
|---|---|---|---|
| α-chain | TSESDYY [SEQ ID | QEAYKQQN [SEQ ID | CAYRSDGGATNKLIF [SEQ ID |
| | NO: 24] | NO: 15] | NO: 25] |
| β-chain | MNHEY [SEQ ID | SMNVEV [SEQ ID | CASSLGAGGYNSPLHF [SEQ ID |
| | NO: 26] | NO: 27] | NO: 28] |

| | |
|---|---|
| α-chain | MACPGFLWALVISTCLEFSMAQTVTQSQPEMSVQEAETVTLSCTYDTSESDYYLFWYKQPPS |
| variable | RQMILVIRQEAYKQQNATENRFSVNFQKAAKSFSLKISDSQLGDAAMYFCAYRSDGGATNKL |
| | IFGTGTLLAVQP [SEQ ID NO: 29] |
| β-chain | MGPQLLGYVVLCLLGAGPLEAQVTQNPRYLITVTGKKLTVTCSQNMNHEYMSWYRQDPGLGL |
| variable | RQIYYSMNVEVTDKGDVPEGYKVSRKEKRNFPLILESPSPNQTSLYFCASSLGAGGYNSPLH |
| | FGNGTRLTVT [SEQ ID NO: 30] |
| Full α- | MACPGFLWALVISTCLEFSMAQTVTQSQPEMSVQEAETVTLSCTYDTSESDYYLFWYKQPPS |
| chain | RQMILVIRQEAYKQQNATENRFSVNFQKAAKSFSLKISDSQLGDAAMYFCAYRSDGGATNKL |
| | IFGTGTLLAVQPNIQNPDPAVYQLRDSKSSDKSVCLFTDFDSQTNVSQSKDSDVYITDKTVL |
| | DMRSMDFKSNSAVAWSNKSDFACANAFNNSIIPEDTFFPSPESSCDVKLVEKSFETDTNLNF |
| | QNLSVIGFRILLLKVAGENLLMTLRLWSS [SEQ ID NO: 31] |
| Full β- | MGPQLLGYVVLCLLGAGPLEAQVTQNPRYLITVTGKKLTVTCSQNMNHEYMSWYRQDPGLGL |
| chain | RQIYYSMNVEVTDKGDVPEGYKVSRKEKRNFPLILESPSPNQTSLYFCASSLGAGGYNSPLH |
| | FGNGTRLTVTEDLNKVEPPEVAVFEPSEAEISHTQKATLVCLATGFFPDHVELSWWVNGKEV |
| | HSGVSTDPQPLKEQPALNDSRYCLSSRLRVSATFWQNPRNHFRCQVQFYGLSENDEWTQDRA |
| | KPVTQIVSAEAWGRADCGFTSVSYQQGVLSATILYEILLGKATLYAVLVSALVLMAMVKRKD |
| | F [SEQ ID NO: 32] |

In certain embodiments, the extracellular domain of the TCR comprises an α chain variable region comprising a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 35 or a conservative modification thereof. SEQ ID NOS: 33-35 are disclosed in Table 4. In certain embodiments, the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35.

In certain embodiments, the extracellular domain of the TCR comprises a β chain variable region comprising a CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof. SEQ ID NOS: 36-38 are disclosed in Table 4. In certain embodiments, the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

In certain embodiments, the α chain variable region comprises a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 35 or a conservative modification thereof; and the β chain variable region CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof In certain embodiments, the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and CDR3 comprising the amino acid sequence set forth in SEQ ID NO: 38. In certain embodiments, the TCR comprises an α chain comprising the amino acid sequence set forth in SEQ ID NO: 39.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 39. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 39. In certain embodiments, the α chain variable region comprises the amino acid sequence set forth in SEQ ID NO: 39. SEQ ID NO: 39 is provided in Table 4.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 40. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 40. In certain embodiments, the β chain variable region comprises the amino acid sequence set forth in SEQ ID NO: 40. SEQ ID NO: 40 is provided in Table 4.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 39; and the β chain variable region comprises an amino acid sequence that is at least about 80% (e.g., at least about 85%, at least about 90%, or at least about 95%) homologous or identical to the amino acid sequence set forth in SEQ ID NO: 40. In certain embodiments, the α chain variable region comprises the amino acid sequence set forth in SEQ ID NO: 39; and the β chain variable region comprises the amino acid sequence set forth in SEQ ID NO: 40.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 41. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 41. In certain embodiments, the α chain comprises the amino acid sequence set forth in SEQ ID NO: 41.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 42. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 42. In certain embodiments, the β chain comprises the amino acid sequence set forth in SEQ ID NO: 42.

In certain embodiments, the α chain comprises an amino acid sequence that is at least about 80% (e.g., at least about 85%, at least about 90%, or at least about 95%) homologous or identical to the amino acid sequence set forth in SEQ ID NO: 41; and the β chain comprises an amino acid sequence that is at least about 80% (e.g., at least about 85%, at least about 90%, or at least about 95%) homologous or identical to the amino acid sequence set forth in SEQ ID NO: 42. In certain embodiments, the α chain comprises the amino acid sequence set forth in SEQ ID NO: 41; and the β chain comprises the amino acid sequence set forth in SEQ ID NO: 42. In certain embodiments, the TCR is designated as “TCR 4”. In certain embodiments, the TCR4 binds to a RAS peptide comprising or consisting of the amino acid sequence set forth in SEQ ID NO: 1. In certain embodiments, the TCR4 binds to a RAS peptide comprising or consisting of the amino acid sequence set forth in SEQ ID NO: 2.

In certain embodiments, the CDRs sequences described above including Table 4 are delineated using the IMGT numbering system.

TABLE 4
(TCR4)

| CDRs | 1 | 2 | 3 |
|---|---|---|---|
| α-chain | NSAFQY [SEQ ID | TYSSGN [SEQ ID | CAMGALNSGAGSYQLTF [SEQ ID |
| | NO: 33] | NO: 34] | NO: 35] |
| β-chain | SGHRS [SEQ ID | YFSETQ [SEQ ID | CASSLSSGTGTEAFF [SEQ ID |
| | NO: 36] | NO: 37] | NO: 38] |

| | |
|---|---|
| α-chain | MMKSLRVLLVILWLQLSWVWSQQKEVEQDPGPLSVPEGAIVSLNCTYSNSAFQYFMWYRQYS |
| variable | RKGPELLMYTYSSGNKEDGRFTAQVDKSSKYISLFIRDSQPSDSATYLCAMGALNSGAGSYQ |
| | LTFGKGTKLSVIP [SEQ ID NO: 39] |
| β-chain | MGSRLLCWVLLCLLGAGPVKAGVTQTPRYLIKTRGQQVTLSCSPISGHRSVSWYQQTPGQGL |
| variable | QFLFEYFSETQRNKGNFPGRESGRQFSNSRSEMNVSTLELGDSALYLCASSLSSGTGTEAFF |
| | GQGTRLTVV [SEQ ID NO: 40] |
| Full α- | MMKSLRVLLVILWLQLSWVWSQQKEVEQDPGPLSVPEGAIVSLNCTYSNSAFQYFMWYRQYS |
| chain | RKGPELLMYTYSSGNKEDGRFTAQVDKSSKYISLFIRDSQPSDSATYLCAMGALNSGAGSYQ |
| | LTFGKGTKLSVIPNIQNPDPAVYQLRDSKSSDKSVCLFTDFDSQTNVSQSKDSDVYITDKTV |
| | LDMRSMDFKSNSAVAWSNKSDFACANAFNNSIIPEDTFFPSPESSCDVKLVEKSFETDTNLN |
| | FQNLSVIGFRILLLKVAGENLLMTLRLWSS [SEQ ID NO: 41] |
| Full β- | MGSRLLCWVLLCLLGAGPVKAGVTQTPRYLIKTRGQQVTLSCSPISGHRSVSWYQQTPGQGL |
| chain | QFLFEYFSETQRNKGNFPGRESGRQFSNSRSEMNVSTLELGDSALYLCASSLSSGTGTEAFF |
| | GQGTRLTVVEDLNKVFPPEVAVFEPSEAEISHTQKATLVCLATGFFPDHVELSWWVNGKEVH |
| | SGVSTDPQPLKEQPALNDSRYCLSSRLRVSATFWQNPRNHFRCQVQFYGLSENDEWTQDRAK |
| | PVTQIVSAEAWGRADCGFTSVSYQQGVLSATILYEILLGKATLYAVLVSALVLMAMVKRKDF |
| | [SEQ ID NO: 42] |

In certain embodiments, the extracellular domain of the TCR comprises an α chain variable region comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOS: 43-45 are disclosed in Table 5. In certain embodiments, the α chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45.

In certain embodiments, the extracellular domain of the TCR comprises a β chain variable region comprising a CDR1 comprising the amino acid sequence set forth in SEQ ID NO: 58 or a conservative modification thereof, a CDR2 comprising the amino acid sequence set forth in SEQ ID NO: 46 or a conservative modification thereof, and a CDR3 comprising the amino acid sequence set forth in SEQ ID NO: 47 or a conservative modification thereof. SEQ ID NOS: 58, 46, and 47 are disclosed in Table 5. In certain embodiments, the f3 chain variable region CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47.

In certain embodiments, the α chain variable region comprises a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and the β chain variable region comprises a CDR1 comprising the amino acid sequence set forth in SEQ ID NO: 58 or a conservative modification thereof, a CDR2 comprising the amino acid sequence set forth in SEQ ID NO: 46 or a conservative modification thereof, and a CDR3 comprising the amino acid sequence set forth in SEQ ID NO: 47 or a conservative modification thereof. In certain embodiments, the α chain variable region comprises a CDR1 comprising the amino acid sequence set forth in SEQ ID NO: 43, a CDR2 comprising the amino acid sequence set forth in SEQ ID NO: 44, a CDR3 comprising the amino acid sequence set forth in SEQ ID NO: 45; and the β chain variable region comprises a CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47. In certain embodiments, the TCR comprises an α chain comprising the amino acid sequence set forth in SEQ ID NO: 48.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 48. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 48. In certain embodiments, the α chain variable region comprises the amino acid sequence set forth in SEQ ID NO: 48. SEQ ID NO: 48 is provided in Table 5.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 49. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 49. In certain embodiments, the β chain variable region comprises the amino acid sequence set forth in SEQ ID NO: 49. SEQ ID NO: 49 is provided in Table 5.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 48; and the β chain variable region comprises an amino acid sequence that is at least about 80% (e.g., at least about 85%, at least about 90%, or at least about 95%) homologous or identical to the amino acid sequence set forth in SEQ ID NO: 49. In certain embodiments, the α chain variable region comprises the amino acid sequence set forth in SEQ ID NO: 48; and the β chain variable region comprises the amino acid sequence set forth in SEQ ID NO: 49.

In certain embodiments, the extracellular domain of the TCR comprises an α chain that comprises an α chain variable region and an α chain constant region. In certain embodiments, the α chain comprises an amino acid sequence that is at least about 80% (e.g., at least about 85%, at least about 90%, or at least about 95%) homologous or identical to the amino acid sequence set forth in SEQ ID NO: 50. For example, the α chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50. In certain embodiments, the α chain comprises the amino acid sequence set forth in SEQ ID NO: 50.

In certain embodiments, the extracellular domain of the TCR comprises a β chain that comprises a β chain variable region and a β chain constant region. In certain embodiments, the β chain comprises an amino acid sequence that is at least about 80% (e.g., at least about 85%, at least about 90%, or at least about 95%) homologous or identical to the amino acid sequence set forth in SEQ ID NO: 51. For example, the β chain comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 51. In certain embodiments, the β chain comprises the amino acid sequence set forth in SEQ ID NO: 51.

In certain embodiments, the α chain comprises an amino acid sequence that is at least about 80% (e.g., at least about 85%, at least about 90%, or at least about 95%) homologous or identical to the amino acid sequence set forth in SEQ ID NO: 50; and the β chain comprises an amino acid sequence that is at least about 80% (e.g., at least about 85%, at least about 90%, or at least about 95%) homologous or identical to the amino acid sequence set forth in SEQ ID NO: 51. In certain embodiments, the α chain comprises the amino acid sequence set forth in SEQ ID NO: 50; and the β chain comprises the amino acid sequence set forth in SEQ ID NO: 51. In certain embodiments, the TCR is designated as “TCR 5”. In certain embodiments, the TCR5 binds to a RAS peptide comprising or consisting of the amino acid sequence set forth in SEQ ID NO: 1. In certain embodiments, the TCR5 binds to a RAS peptide comprising or consisting of the amino acid sequence set forth in SEQ ID NO: 2.

In certain embodiments, the CDRs sequences described above including Table 5 are delineated using the IMGT numbering system.

TABLE 5
(TCR5)

| CDRs | 1 | 2 | 3 |
|---|---|---|---|
| α-chain | DSSSTY [SEQ ID | IFSNMDM [SEQ ID | CAERDAGNNRKLIW [SEQ ID NO: |
| | NO: 43] | NO: 44] | 45] |
| β-chain | SGHVS [SEQ ID | FQNEAQ [SEQ ID | CASSLEGGDTQYF [SEQ ID NO: |
| | NO: 58] | NO: 46] | 47] |

| | |
|---|---|
| α-chain | MKTFAGFSFLFLWLQLDCMSRGEDVEQSLFLSVREGDSSVINCTYTDSSSTYLYWYKQEPGA |
| variable | GLQLLTYIFSNMDMKQDQRLTVLLNKKDKHLSLRIADTQTGDSAIYFCAERDAGNNRKLIWG |
| | LGTSLAVNP [SEQ ID NO: 48] |
| β-chain | MGTRLLCWVVLGFLGTDHTGAGVSQSPRYKVAKRGQDVALRCDPISGHVSLFWYQQALGQGP |
| variable | EFLTYFQNEAQLDKSGLPSDRFFAERPEGSVSTLKIQRTQQEDSAVYLCASSLEGGDTQYFG |
| | PGTRLTVL [SEQ ID NO: 49] |
| Full α- | MKTFAGFSFLFLWLQLDCMSRGEDVEQSLFLSVREGDSSVINCTYTDSSSTYLYWYKQEPGA |
| chain | GLQLLTYIFSNMDMKQDQRLTVLLNKKDKHLSLRIADTQTGDSAIYFCAERDAGNNRKLIWG |
| | LGTSLAVNPNIQNPDPAVYQLRDSKSSDKSVCLFTDFDSQTNVSQSKDSDVYITDKTVLDMR |
| | SMDFKSNSAVAWSNKSDFACANAFNNSIIPEDTFFPSPESSCDVKLVEKSFETDTNLNFQNL |
| | SVIGFRILLLKVAGENLLMTLRLWSS [SEQ ID NO: 50] |
| Full β- | MGTRLLCWVVLGFLGTDHTGAGVSQSPRYKVAKRGQDVALRCDPISGHVSLFWYQQALGQGP |
| chain | EFLTYFQNEAQLDKSGLPSDRFFAERPEGSVSTLKIQRTQQEDSAVYLCASSLEGGDTQYFG |
| | PGTRLTVLEDLKNVFPPEVAVFEPSEAEISHTQKATLVCLATGFYPDHVELSWWVNGKEVHS |
| | GVSTDPQPLKEQPALNDSRYCLSSRLRVSATFWQNPRNHERCQVQFYGLSENDEWTQDRAKP |
| | VTQIVSAEAWGRADCGFTSESYQQGVLSATILYEILLGKATLYAVLVSALVLMAMVKRKDSR |
| | G [SEQ ID NO: 51] |

In certain embodiments, the α chain variable region and/or the β chain variable region amino acid sequences have at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NO: 10, SEQ ID NO: 11, SEQ ID NO: 20, SEQ ID NO: 21, SEQ ID NO: 29, SEQ ID NO: 30, SEQ ID NO: 39, SEQ ID NO: 40, SEQ ID NO: 48, and SEQ ID NO: 49) comprise modifications, including, but not limited to, substitutions (e.g., conservative substitutions), insertions, or deletions relative to the specified sequence(s), but retain the ability to bind to a mutant RAS peptide (e.g., a G12D mutant RAS peptide). In certain embodiments, such modifications are not within the CDR domains of the variable regions.

In certain embodiments, a total of 1 to 10 amino acids are substituted, inserted and/or deleted in SEQ ID NO: 10, SEQ ID NO: 11, SEQ ID NO: 20, SEQ ID NO: 21, SEQ ID NO: 29, SEQ ID NO: 30, SEQ ID NO: 39, SEQ ID NO: 40, SEQ ID NO: 48, or SEQ ID NO: 49. In certain embodiments, substitutions, insertions, or deletions occur in regions outside the CDRs of the extracellular domain. In certain embodiments, the extracellular domain comprises an α chain variable region and/or a β chain variable region sequence selected from the group consisting of SEQ ID NO: 10, SEQ ID NO: 11, SEQ ID NO: 20, SEQ ID NO: 21, SEQ ID NO: 29, SEQ ID NO: 30, SEQ ID NO: 39, SEQ ID NO: 40, SEQ ID NO: 48, and SEQ ID NO: 49, including post-translational modifications of that sequence (SEQ ID NO: 10, SEQ ID NO: 11, SEQ ID NO: 20, SEQ ID NO: 21, SEQ ID NO: 29, SEQ ID NO: 30, SEQ ID NO: 39, SEQ ID NO: 40, SEQ ID NO: 48, or SEQ ID NO: 49).

### 5.3.1.2. Constant Regions

In certain embodiments, the presently disclosed TCR comprises an α chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 53 or SEQ ID NO: 54. In certain embodiments, the α chain constant region comprises the amino acid sequence set forth in SEQ ID NO: 53. In certain embodiments, the α chain constant region comprises the amino acid sequence set forth in SEQ ID NO: 54.

In certain embodiments, a TCR disclosed herein comprises a β chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 55, SEQ ID NO: 56, SEQ ID NO: 57. In certain embodiments, the β chain constant region comprises the amino acid sequence set forth in SEQ ID NO: 55. In certain embodiments, the β chain constant region comprises the amino acid sequence set forth in SEQ ID NO: 56. In certain embodiments, the β chain constant region comprises the amino acid sequence set forth in SEQ ID NO: 57. SEQ ID NOS: 53-57 are provided below:

Human α chain constant region:

[SEQ ID NO: 53]
NIQNPDPAVYQLRDSKSSDKSVCLFTDFDSQTNVSQSKDS
DVYITDKTVLDMRSMDFKSNSAVAWSNKSDFACANAFNNS
IIPEDTFFPSPESSCDVKLVEKSFETDTNLNFQNLSVIGF
RILLLKVAGENLLMTLRLWSS

Mouse α chain constant region (cysteine-modification and LVL modification in transmembrane domain underlined):

[SEQ ID NO: 54]
NIQNPEPAVYQLKDPRSQDSTLCLFTDFDSQINVPKTMES
GTFITDKCVLDMKAMDSKSNGAIAWSNQTSFTCQDIFKET
NATYPSSDVPCDATLTEKSFETDMNLNFQNLLVIVLRILL
LKVAGENLLMTLRLWSS

Human β chain constant region:

[SEQ ID NO: 55]
EDLNKVFPPEVAVFEPSEAEISHTQKATLVCLATGFFPDH
VELSWWVNGKEVHSGVSTDPQPLKEQPALNDSRYCLSSRL
RVSATFWQNPRNHFRCQVQFYGLSENDEWTQDRAKPVTQI
VSAEAWGRADCGFTSVSYQQGVLSATILYEILLGKATLYA
VLVSALVLMAMVKRKDF

Mouse β chain constant region (cysteine-modification underlined):

[SEQ ID NO: 56]
EDLRNVTPPKVSLFEPSKAEIANKQKATLVCLARGFFPDH
VELSWWVNGKEVHSGVCTDPQAYKESNYSYCLSSRLRVSA
TFWHNPRNHFRCQVQFHGLSEEDKWPEGSPKPVTQNISAE
AWGRADCGITSASYQQGVLSATILYEILLGKATLYAVLVS
TLVVMAMVKRKNS

Human β chain constant region:

[SEQ ID NO: 57]
EDLKNVFPPEVAVFEPSEAEISHTQKATLVCLATGFYPDH
VELSWWVNGKEVHSGVSTDPQPLKEQPALNDSRYCLSSRL
RVSATFWQNPRNHFRCQVQFYGLSENDEWTQDRAKPVTQI
VSAEAWGRADCGFTSESYQQGVLSATILYEILLGKATLYA
VLVSALVLMAMVKRKDSRG

5.3.2. TCRs that Bind to the Same RAS Peptide as TCR clonotypes

The presently disclosed subject matter further provides TCRs that bind to the same RAS peptide (e.g., a G12D mutant RAS peptide) as a TCR disclosed herein (e.g., a TCE disclosed in Section 5.3.1). In certain embodiments, the TCR binds to the same RAS peptide (e.g., a G12D mutant RAS peptide) as a reference TCR or a functional fragment thereof comprising the α chain variable region CDR1, CDR2, and CDR3 sequences and the β chain variable region CDR1, CDR2, and CDR3 sequences of, for example, any one of the TCRs disclosed herein (e.g., those disclosed in Section 5.3.1). In certain embodiments, the TCR binds to the same RAS peptide (e.g., a G12D mutant RAS peptide) as a reference TCR or a functional fragment thereof comprising the α chain variable region and the β chain variable region sequences of, for example, any one of the presently disclosed TCRs (e.g., those disclosed in Section 5.3.1).

### 5.3.3. TCRs Having Specific CDR3 Sequences

It is well known in the art that the CDR3 domain, independently from the CDR1 and/or CDR2 domain(s), alone can determine the binding specificity of a TCR or a functional fragment thereof, for a cognate antigen and that multiple TCRs can predictably be generated having the same binding specificity based on a common CDR3 sequence.

In certain embodiments, the extracellular domain of the TCR comprises an α chain variable region CDR3 comprising the amino acid sequence set forth in SEQ ID NO: 6 or a conservative modification thereof; and a β chain variable region CDR3 comprising the amino acid sequence set forth in SEQ ID NO: 9 or a conservative modification thereof. In certain embodiments, the extracellular domain of the TCR further comprises an α chain variable region CDR2 comprising the amino acid sequence set forth in SEQ ID NO: 5 or a conservative modification thereof; and a β chain variable region CDR2 comprising the amino acid sequence set forth in SEQ ID NO: 8 or a conservative modification thereof. In certain embodiments, the extracellular domain of the TCR further comprise san α chain variable region CDR1 comprising the amino acid sequence set forth in SEQ ID NO: 4 or a conservative modification thereof; and a β chain variable region CDR1 comprising the amino acid sequence set forth in SEQ ID NO: 7 or a conservative modification thereof.

In certain embodiments, the extracellular domain of the TCR comprises an α chain variable region CDR3 comprising the amino acid sequence set forth in SEQ ID NO: 16 or a conservative modification thereof; and a β chain variable region CDR3 comprising the amino acid sequence set forth in SEQ ID NO: 19 or a conservative modification thereof In certain embodiments, the extracellular domain of the TCR further comprises an α chain variable region CDR2 comprising the amino acid sequence set forth in SEQ ID NO: 15 or a conservative modification thereof; and a β chain variable region CDR2 comprising the amino acid sequence set forth in SEQ ID NO: 18 or a conservative modification thereof. In certain embodiments, the extracellular domain of the TCR further comprise an α chain variable region CDR1 comprising the amino acid sequence set forth in SEQ ID NO: 14 or a conservative modification thereof; and a β chain variable region CDR1 comprising the amino acid sequence set forth in SEQ ID NO: 17 or a conservative modification thereof.

In certain embodiments, the extracellular domain of the TCR comprises an α chain variable region CDR3 comprising the amino acid sequence set forth in SEQ ID NO: 25 or a conservative modification thereof; and a β chain variable region CDR3 comprising the amino acid sequence set forth in SEQ ID NO: 28 or a conservative modification thereof. In certain embodiments, the extracellular domain of the TCR further comprises an α chain variable region CDR2 comprising the amino acid sequence set forth in SEQ ID NO: 15 or a conservative modification thereof; and a β chain variable region CDR2 comprising the amino acid sequence set forth in SEQ ID NO: 27 or a conservative modification thereof. In certain embodiments, the extracellular domain of the TCR further comprise an α chain variable region CDR1 comprising the amino acid sequence set forth in SEQ ID NO: 24 or a conservative modification thereof; and a β chain variable region CDR1 comprising the amino acid sequence set forth in SEQ ID NO: 26 or a conservative modification thereof.

In certain embodiments, the extracellular domain of the TCR comprises an α chain variable region CDR3 comprising the amino acid sequence set forth in SEQ ID NO: 35 or a conservative modification thereof; and a β chain variable region CDR3 comprising the amino acid sequence set forth in SEQ ID NO: 38 or a conservative modification thereof. In certain embodiments, the extracellular domain of the TCR further comprises an α chain variable region CDR2 comprising the amino acid sequence set forth in SEQ ID NO: 34 or a conservative modification thereof; and a β chain variable region CDR2 comprising the amino acid sequence set forth in SEQ ID NO: 37 or a conservative modification thereof. In certain embodiments, the extracellular domain of the TCR further comprise an α chain variable region CDR1 comprising the amino acid sequence set forth in SEQ ID NO: 33 or a conservative modification thereof; and a β chain variable region CDR1 comprising the amino acid sequence set forth in SEQ ID NO: 36 or a conservative modification thereof.

In certain embodiments, the extracellular domain of the TCR comprises an α chain variable region CDR3 comprising the amino acid sequence set forth in SEQ ID NO: 45 or a conservative modification thereof; and a β chain variable region CDR3 comprising the amino acid sequence set forth in SEQ ID NO: 47 or a conservative modification thereof. In certain embodiments, the extracellular domain of the TCR further comprises an α chain variable region CDR2 comprising the amino acid sequence set forth in SEQ ID NO: 44 or a conservative modification thereof; and a β chain variable region CDR2 comprising the amino acid sequence set forth in SEQ ID NO: 46 or a conservative modification thereof. In certain embodiments, the extracellular domain of the TCR further comprise an α chain variable region CDR1 comprising the amino acid sequence set forth in SEQ ID NO: 43 or a conservative modification thereof; and a β chain variable region CDR1 comprising the amino acid sequence set forth in SEQ ID NO: 58 or a conservative modification thereof.

### 5.3.4. TCRs With Modifications Within CDRs

In certain embodiments, a presently disclosed TCR (or a functional fragment thereof) comprises an α chain variable region comprising CDR1, CDR2 and CDR3 sequences and a β chain variable region comprising CDR1, CDR2 and CDR3 sequences, wherein one or more of these CDR sequences comprise specified amino acid sequences based on the TCRs (or a functional fragments thereof) described herein (see Tables 1-5), or modifications thereof, and wherein the TCRs (or a functional fragments thereof) retain the desired functional properties of the mutant RAS peptide-specific TCRs (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCRs (or a functional fragments thereof) described herein (see Tables 1-5), or modifications thereof, and wherein the TCR (or a functional fragment thereof) retains the desired functional properties of the mutant RAS peptide-specific TCRs (or a functional fragments thereof) of the presently disclosed subject matter.

In certain embodiments, such modifications do not significantly affect or alter the binding characteristics of the TCR comprising the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 6. In certain embodiments, amino acid substitutions may be introduced into a TCR of interest and the products screened for a desired activity, e.g., retained/improved antigen binding, decreased immunogenicity, or improved ADCC or CDC.

TABLE 6

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006; 66(17):8878-8886; Cohen et al. Enhanced antitumor activity of T cells engineered to express T-cell receptors with a second disulfide bond, Cancer Res. 2007; 67(8):3898-3903; Kuball et al. Facilitating matched pairing and expression of TCR chains introduced into human T cells, Blood 2007; 109(6):2331-2338; Haga-Friedman et al. Incorporation of transmembrane hydrophobic mutations in the TCR enhance its surface expression and T cell functional avidity, Journal of immunology 2012; 188(11):5538-5546, the contents of each of which are incorporated by reference in their entireties).

### 5.3.5. Bispecific Molecules

The presently disclosed subject matter provides bispecific molecules comprising a presently disclosed TCR (or a functional fragment thereof). A presently disclosed TCR or a functional fragment thereof can be derivatized or linked to another functional molecule, e.g., another peptide or protein (e.g., another antibody or ligand for a receptor) to generate a bispecific molecule that binds to at least two different binding sites or target molecules. The presently disclosed TCR or a functional fragment thereof can in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, a presently disclosed TCR or a functional fragment thereof can be functionally linked (e.g., by chemical coupling, genetic fusion, noncovalent association or otherwise) to one or more other binding molecules, such as another antibody, antibody fragment, peptide or binding mimetic.

The presently disclosed subject matter provides bispecific molecules comprising at least a first binding specificity for a mutant RAS peptide and a second binding specificity for a second target peptide region. The second target epitope region can be a second RAS peptide, or a non-RAS peptide, e.g., a different antigen. In certain embodiments, the bispecific molecule is multi-specific, e.g., the molecule can further include a third binding specificity. Where a first portion of a bispecific molecule, e.g., antibody, binds to an antigen on a tumor cell for example and a second portion of a bispecific molecule recognizes an antigen on the surface of a human immune effector cell, the bispecific molecule is capable of recruiting the activity of that effector cell by specifically binding to the effector antigen on the human immune effector cell. In certain embodiments, bispecific molecules are able to form a link between effector cells, for example, T cells and tumor cells, thereby enhancing effector function. In certain embodiments, a presently disclosed bispecific molecule comprises at least a first binding to a mutant RAS peptide and at least a second binding to an immune cell or a molecule associated with an immune cell.

The bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, MA et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb and a mAb, a mAb and a Fab, a Fab and a F(ab′)₂, or a ligand and a Fab fusion protein.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a y counter or a scintillation counter or by autoradiography.

### 5.4. Cells

The presently disclosed subject matter provides cells comprising a presently disclosed TCR (e.g., one disclosed in Section 5.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells and/or stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the TCR-expressing T cells express Foxp3 to achieve and maintain a T regulatory phenotype.

In certain embodiments, the T cell is a NK-T cell. Natural killer (NK) T cells can be lymphocytes that are part of cell-mediated immunity and act during the innate immune response. NK-T cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000);164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, cell further comprises at least one recombinant or exogenous co-stimulatory ligand. For example, a presently disclosed cell can be further transduced with at least one co-stimulatory ligand, such that the cell co-expresses or is induced to co-express the presently disclosed TCR and the at least one co-stimulatory ligand. The interaction between the presently disclosed TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX4OL/CD252, CD27L/CD70, Fas ligand (FasL), CD30L/CD153, tumor necrosis factor beta (TNF-62 )/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof In certain embodiments, the cell comprises one recombinant co-stimulatory ligand that is 4-1BBL. In certain embodiments, the cell comprises two recombinant co-stimulatory ligands that are 4-1BBL and CD80.

In certain embodiments, a presently disclosed cell further comprises at least one exogenous cytokine. For example, a presently disclosed cell can be further transduced with at least one cytokine, such that the cell secretes the at least one cytokine as well as expresses the presently disclosed TCR. In certain embodiments, the at least one cytokine is selected from the group consisting of IL-2, IL-3, IL-6, IL-7, IL-11, IL-12, IL-15, IL-17, IL-18, and IL-21. In certain embodiments, the cytokine is IL-12.

### 5.5. Nucleic Acids and Genetic Modifications of Cells

The present discloses subject matter provides a nucleic acid encoding a presently disclosed TCR (e.g., one disclosed in Section 5.3). Further provided are cells comprising such nucleic acids. In certain embodiments, a promoter is operably linked to the presently disclosed TCR.

In certain embodiments, the promoter is endogenous or exogenous. In certain embodiments, the exogenous promoter is selected from the group consisting of a long terminal repeat (LTR) promoter, an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiment, the exogenous promoter is a LTR promoter. In certain embodiments, the promoter is an inducible promoter. In certain embodiment, the inducible promoter is selected from the group consisting of a NFAT transcriptional response element (TRE) promoter, a CD69 promoter, a CD25 promoter, and an IL-2 promoter.

In certain embodiments, the nucleic acid encodes both the α chain and the β chain of a presently disclosed TCR. In certain embodiments, the α chain and the β chain are separated by a self-cleavage peptide, e.g., a 2A-peptide. In certain embodiments, the αchain and the β chain are separated by a furin-2A-peptide. In certain embodiments, the peptide comprises the amino acid sequence set forth in SEQ ID NO: 52.

[SEQ ID NO: 52]
RAKRSGSGATNFSLLKQAGDVEENPGP

In certain embodiments, the nucleic acid encodes a functional portion/fragment of a presently disclosed TCR. As used herein, the term “functional portion” or “functional fragment” refers to any portion, part or fragment of a presently disclosed TCR, which portion, part or fragment retains the biological activity of the TCR (the parent TCR). For example, functional portions encompass the portions, parts or fragments of a presently disclosed TCR that retains the ability to recognize the RAS peptide (e.g., a RAS peptide comprising a G12D mutation) to a similar, same, or even a higher extent as the parent TCR. In certain embodiments, the nucleic acid encoding a functional portion of a presently disclosed TCR encodes a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Genetic modification of a cell (e.g., a T cell) can be accomplished by transducing a substantially homogeneous cell composition with a recombinant DNA or RNA construct. In certain embodiments, a retroviral vector (e.g., gamma-retroviral vector or lentiviral vector) is employed for the introduction of the DNA or RNA construct into the cell. For example, a polynucleotide encoding a presently disclosed TCR can be cloned into a retroviral vector and expression can be driven from its endogenous promoter, from the retroviral long terminal repeat, or from an alternative internal promoter, or from a promoter specific for a target cell type of interest. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used. For initial genetic modification of a cell to include a presently disclosed TCR, a retroviral vector can be employed for transduction, however any other suitable viral vector or non-viral delivery system can be used. The TCR can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF -2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides , e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985);5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

In certain embodiments, a presently disclosed TCR can be integrated into a selected locus of the genome of a cell. Any targeted genome editing methods can also be used to deliver a presently disclosed TCR to a cell or a subject. In certain embodiments, a CRISPR system is used to deliver a presently disclosed TCR. In certain embodiments, zinc-finger nucleases are used to deliver presently disclosed TCR. In certain embodiments, a TALEN system is used to deliver a presently disclosed TCR.

In certain embodiments, a presently disclosed TCR can be integrated at a locus encoding a T cell receptor. Non-limiting examples of the loci include a TRAC locus, a TRBC locus, a TRDC locus, and a TRGC locus. In certain embodiments, the locus is a TRAC locus or a TRBC locus. Methods of targeting a TCR to a site within the genome of T cell can be found in WO2017180989 and Eyquem et al., Nature. (2017 Mar. 2); 543(7643): 113-117, both of which are incorporated by reference in their entireties. In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of a cell. In certain embodiments, the CRISPR system uses a DNA donor-template guided homology directed repair at a defined genetic locus, e.g., a TRAC locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. In certain embodiments, CRISPR/Cas9 is a recombinant ribonucleoprotein complex that is transfected into target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of a cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR) In certain embodiments, the modification is introduced downstream of the protein coding region of the integrated TCR.

### 5.6. Formulations and Administration

The presently disclosed subject matter also provides compositions comprising the presently disclosed cells (e.g., those disclosed in Section 5.4). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a tumor. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹¹, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, at least about 1×10⁵ cells can be administered, eventually reaching about 1×10¹⁰ or more. In certain embodiments, at least about 1×10⁶ cells can be administered. In certain embodiments, from about 10⁴ to about 10¹¹, from about 10⁵ to about 10⁹, or from about 10⁶ to about 10⁸ the presently disclosed cells are administered to a subject. More effective cells may be administered in even smaller numbers. In certain embodiments, at least about 1×10⁸, about 2×10⁸, about 3×10⁸, about 4×10⁸, and about 5×10⁸ the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor.

In certain embodiments, each of the above-noted methods comprises administering the presently disclosed cells or a composition (e.g., a pharmaceutical composition) comprising thereof to achieve the desired effect, e.g., palliation of an existing condition or prevention of recurrence of tumor. For treatment, the amount administered is an amount effective in producing the desired effect. An effective amount can be provided in one or a series of administrations. An effective amount can be provided in a bolus or by continuous perfusion.

In certain embodiments, the tumor is associated with RAS. In certain embodiments, the tumor is associated with a RAS mutation or a RAS mutant. In certain embodiments, the RAS mutation is a G12 mutation. In certain embodiments, the RAS mutation is a G12D mutation.

In certain embodiments, the tumor is a cancer. In certain embodiments, the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer (also known as “stomach cancer”), head and neck squamous cell carcinoma, nonmelanoma skin cancer, salivary gland cancer, melanoma, and multiple myeloma. In certain embodiments, the cancer is pancreatic cancer.

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*31, HLA-A*33, HLA-A*66, HLA-A*68 and HLA-A*74. In certain embodiments, the HLA-A*03 superfamily member is HLA-A*11.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

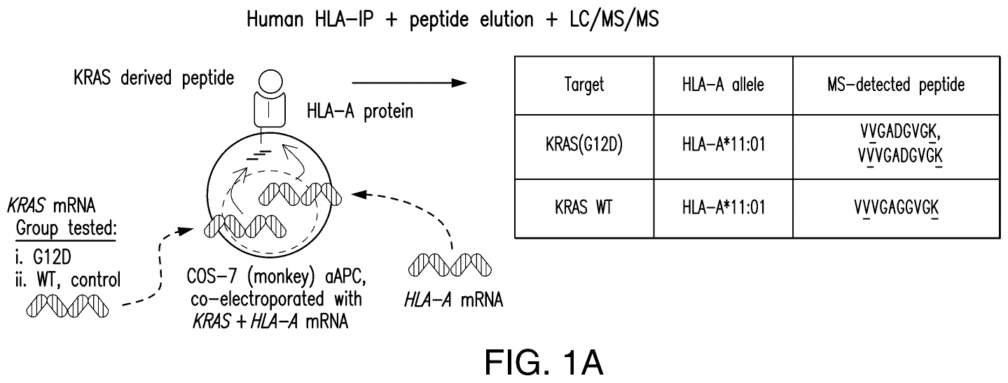

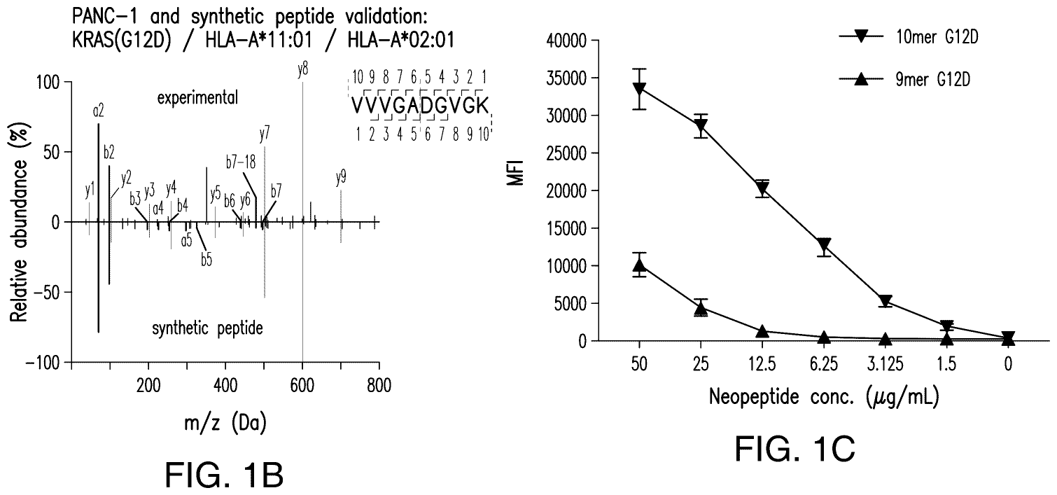

To identify naturally processed and presented epitope(s) resulting from KRAS, COS-7 was used as an artificial antigen presenting cell (aAPC). COS-7 cells were co-electroporated with mRNA encoding HLA-A*11:01 and either full-length KRAS(G12D) or Wild type (WT) KRAS. HLA-restricted immunopeptidome of endogenously processed and presented “public” neoantigens (NeoAgs) resulting from mutant KRAS proteins were screened using HLA immune-precipitation (IP) and tandem mass spectrometry (MS/MS). FIG. 1A shows a table summarizing IP/MS-MS screen-detected peptides resulting from the KRAS proteins. Both 10mer and 9mer peptides encompassing the (G12D) hotspot mutation were detected. The 10-mer WT variant was also detected. PANC-1 cells naturally express KRAS(G12D) and are HLA-A*11:01⁺ and HLA-A*02:01⁺. FIG. 1B shows a validation MS “mirror” plot for an eluted HLA-A*11:01-restricted KRAS(G12D) peptide from a PANC-1 pancreatic cancer cell line (top) and a confirmatory synthetic peptide (bottom). FIG. 1C shows assessment of the stability of neopeptide/HLA complex on the cell surface. T2 cells, a TAP-deficient cell line, were electroporated with HLA-A*11:01 and pulsed with titrating amounts of KRAS(G12D) 9-mer and 10-mer neopeptide variants. Cell surface expression of HLA-A*11:01 was measured by flow cytometry as a correlate of p/HLA complex stability.

As shown in FIG. 2, all four members of the RAS family share 90% sequence homology throughout their G domains but differ significantly in their N-terminal membrane targeting domains. Of note, the amino acid sequences surrounding the codon 12 hotspot region share 100% sequence homology between RAS family members. This suggests that a TCR specific for KRAS(G12D) might afford cross-protection to other mutant RAS proteins.

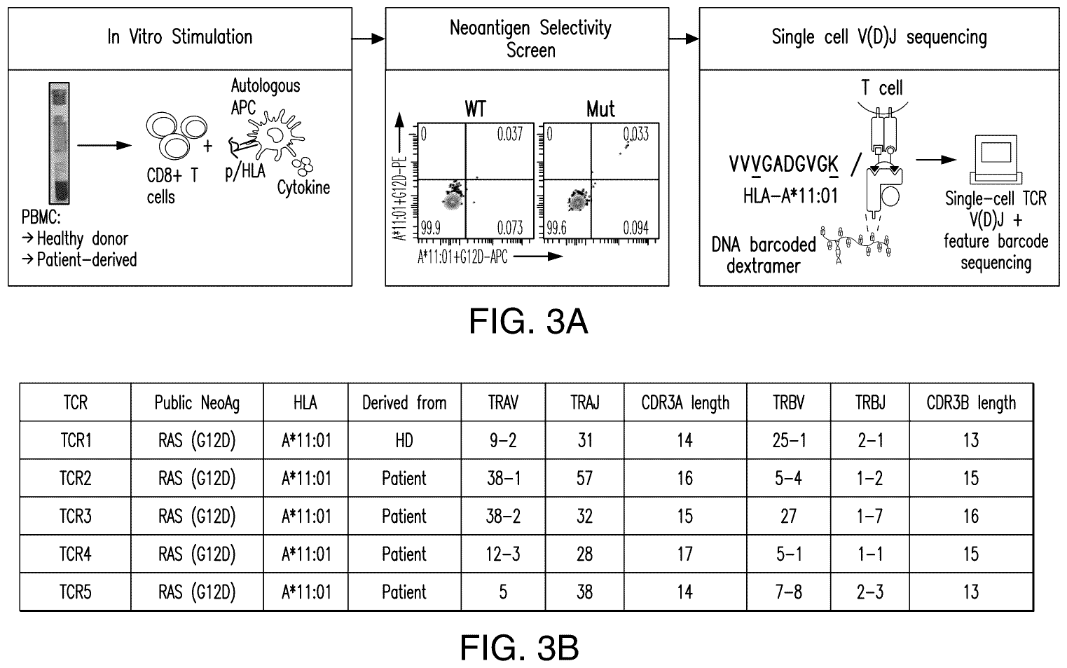

Next, studies were conducted to discover unique HLA-A*11:01-restricted RAS-specific TCR clonotypes. As shown in FIG. 3A, T cells derived from HLA-A*11:01+ healthy-donors (HDs) or HLA-A*11:01+ patients with a history of a KRAS(G12D) cancer were stimulated in vitro with autologous antigen presenting cells presenting KRAS(G12D). Individual cultures were screened for the presence of RAS-specific T cells using a higher-order peptide/HLA dextramer reagent loaded with the mass-spec identified 10mer epitope. Positive wells were labeled with barcoded-dextramers and subjected to combined single-cell V(D)J and feature barcode sequencing to retrieve TCR gene sequences of RAS-specific T cell clonotypes. Five unique RAS-specific TCRs were retrieved from a healthy-donor (n=1) and patient-derived samples (n=4). As depicted in FIG. 3B, all five TCRs were composed of unique alpha and beta variable chain segments and CDR3 loop lengths.

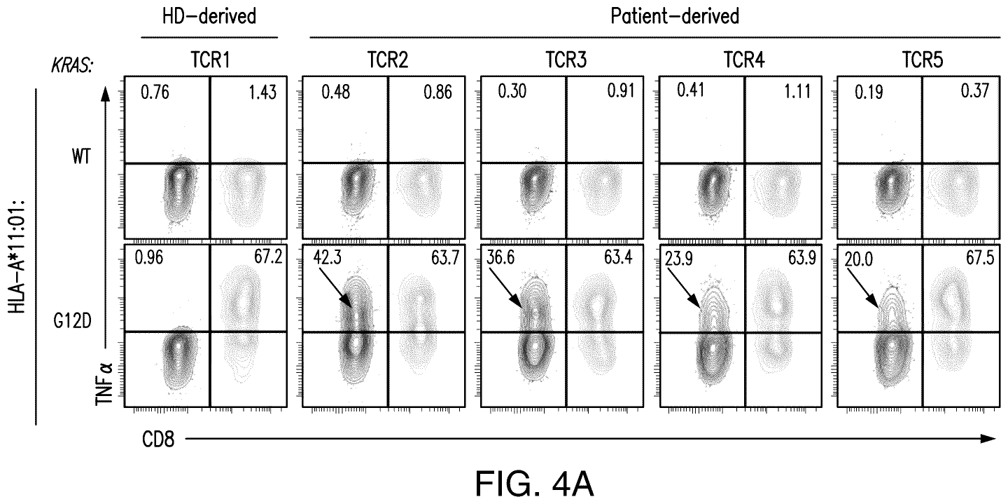

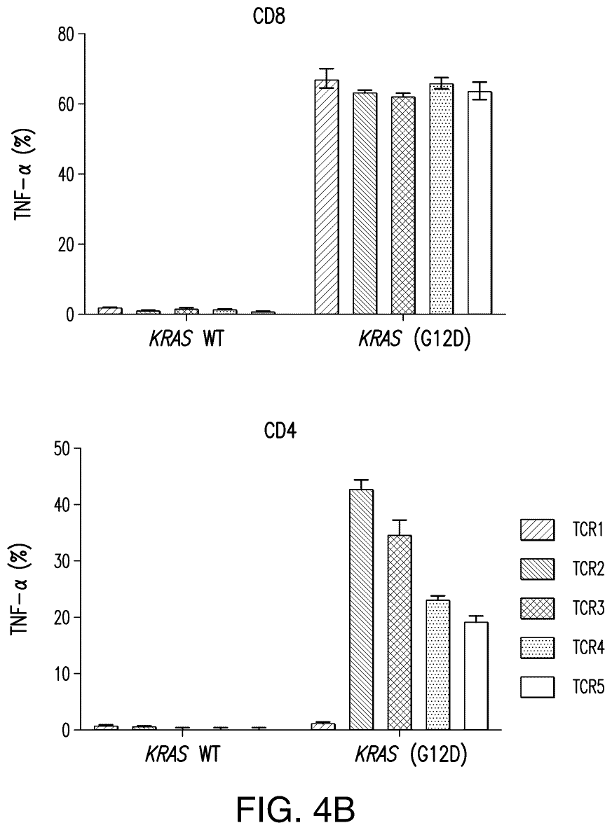

Next, studies were conducted to functionally validate and measure co-receptor dependency of healthy donor (HD) and patient-derived TCRs specific for a RAS(G12D) public NeoAg. Open repertoire (non-specific) T cells were retrovirally transduced with an individual retrieved TCR gene sequence. The function of TCR transduced T cells was measured by coculturing with HLA-A*11:01+ target cells co-transfected with mRNA encoding either full-length wild type (WT) KRAS or KRAS(G12D). Intracellular TNF-α production was determined in CD4+ (blue) and CD8+ (red) T cells expressing the transduced TCR. As shown in FIGS. 4A and 4B, all four patient-derived TCRs demonstrated coreceptor-independence.

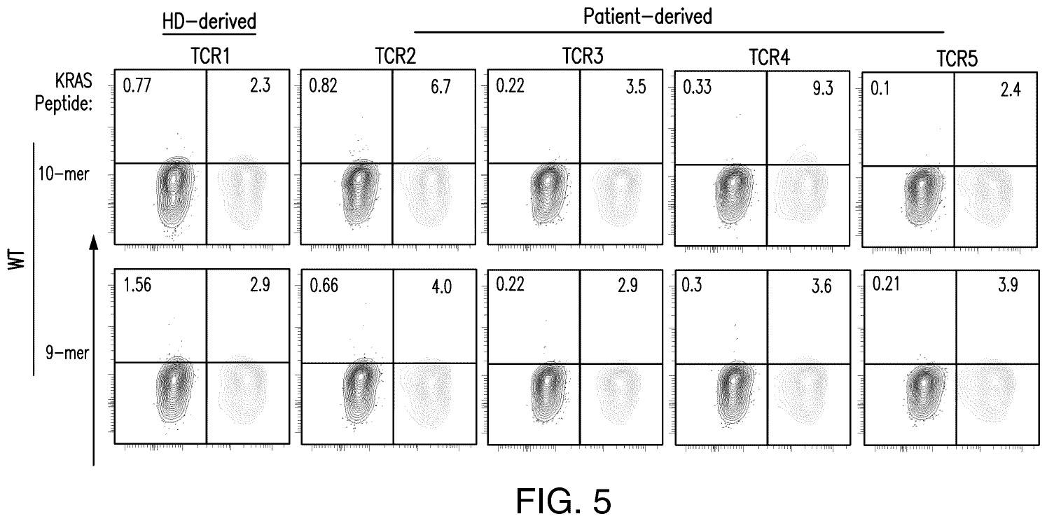

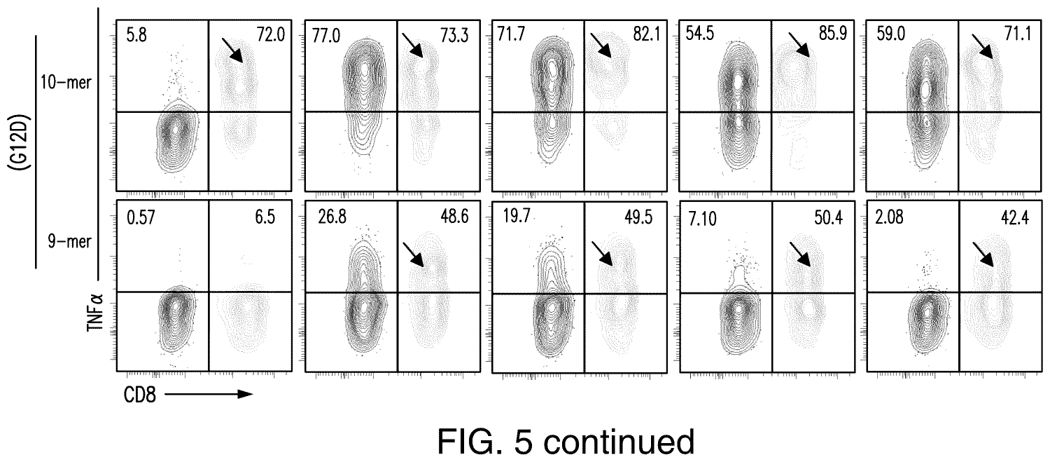

To determine the minimal epitope of individual RAS(G12D)-specific TCR library members, open repertoire T cells were retrovirally transduced with an individual retrieved TCR gene sequence. TCR transduced T cells were cocultured with HLA-A*11:01⁺ target cells pulsed with (10 μg/mL) either the 9-mer or 10-mer neoepitopes derived from KRAS(G12D) or corresponding WT counterparts. Intracellular TNF-α production was determined in CD4⁺ (blue) and CD8⁺ (red) T cells expressing the transduced TCR. As shown by the FACS plots in FIG. 5, the 10mer neopeptide was recognized by all TCR library members, but recognition of the 9mer neoepitope was restricted to the patient-derived TCRs alone.

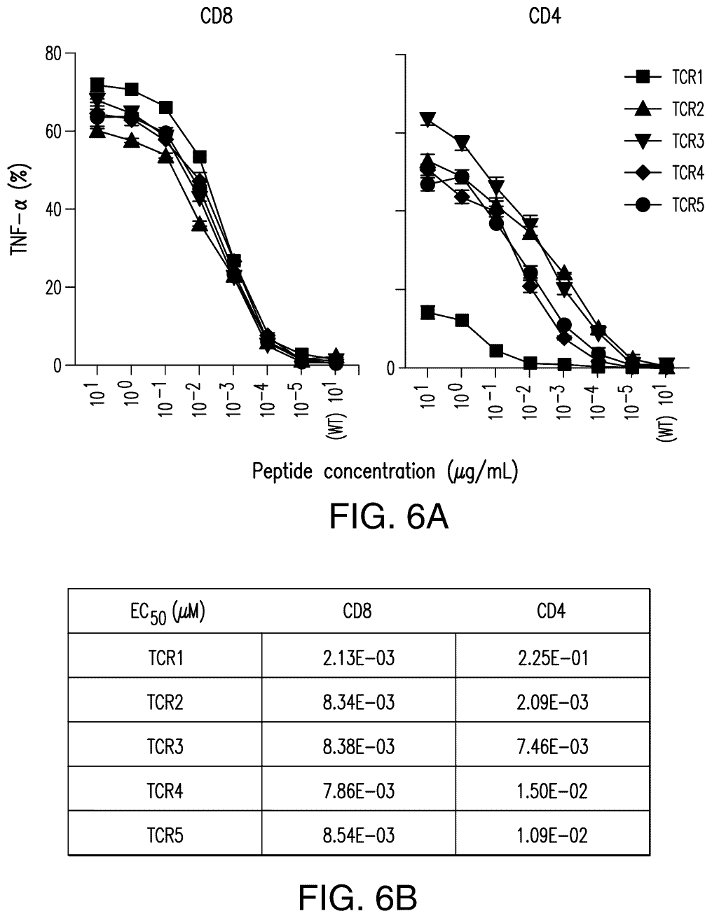

Based on these data, functional avidity of T cells transduced with RAS-specific TCRs was determined. Open-repertoire T cells were retrovirally transduced with an individual retrieved TCR gene sequence and cocultured with HLA-A*11:01⁺ targets pulsed with titrating amounts of 10-mer RAS(G12D) neopeptide. WT peptide was included as a control (10 μg/mL). Intracellular TNF-α production was determined in CD8⁺ (left) and CD4+ (right) TCR+ T cells and is shown in FIG. 6A. EC₅₀ values for each individual TCR in CD8⁺ and CD4⁺ T cells are listed in FIG. 6B.

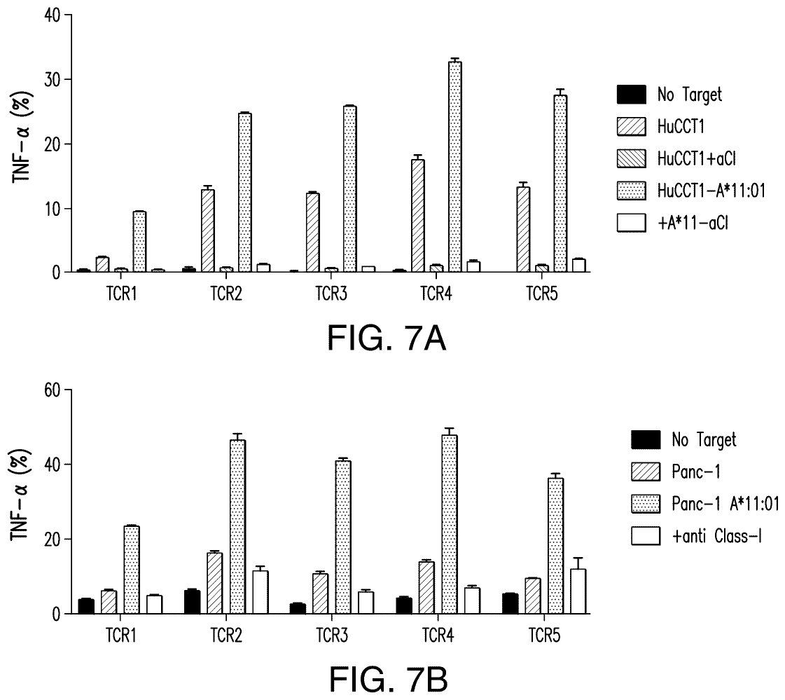

Next, studies were conducted to evaluate the recognition of endogenous levels of KRAS(G12D) in two HLA-A*11:01+ tumor lines by RAS-specific TCR library members. Open-repertoire T cells were retrovirally transduced with an individual retrieved TCR gene sequence and cocultured with either HuCCT1 (FIG. 7A) or PANC-1 (FIG. 7B) cells in the presence or absence of a pan HLA class-I blocking antibody. HuCCT1 is a cholangiocarcinoma line and PANC-1 is a pancreatic tumor line that are both HLA-A*11:01+ and mutant KRAS(G12D). T cells alone were included as a biological control to measure baseline T cell cytokine levels. Intracellular TNF-α production was determined in CD8+ TCR+ T cells and is shown in FIG. 7. TCR library members were capable of recognizing endogenously processed and presented RAS(G12D) levels in a class-I restricted manner.

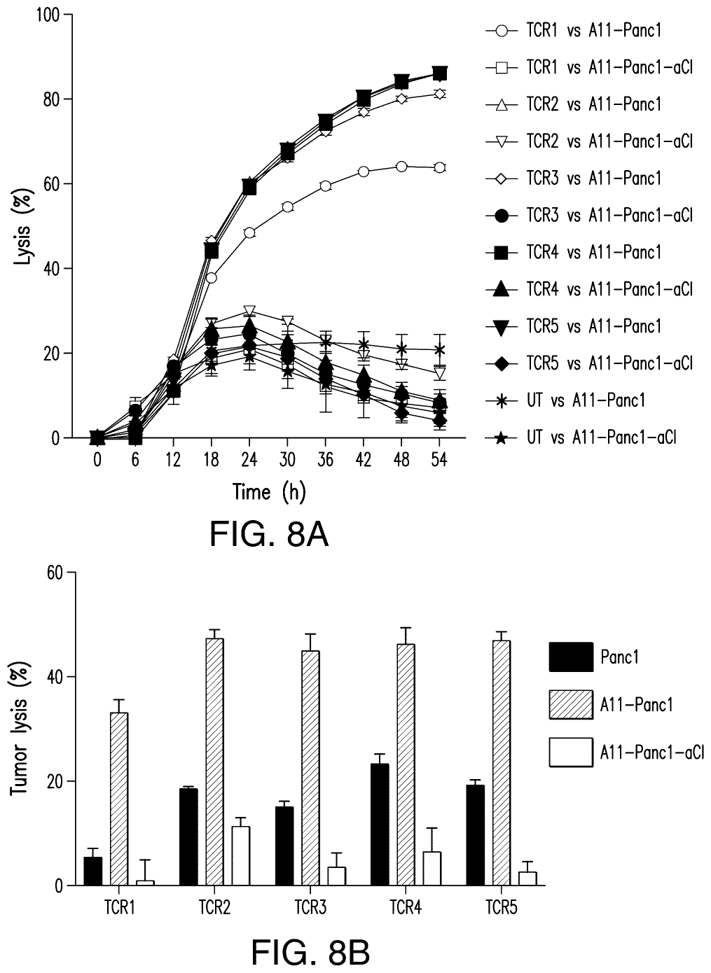

In order to measure the cytolytic capabilities of individual library members, TCR transduced T cells were cocultured with PANC1 in the presence or absence of a pan Class-I blocking antibody. Cytolysis was measured using a tumor impedance based assay over a 54 h time period. FIG. 8A shows tumor curves for individual library members. FIG. 8B shows peak cytolysis at 48 h post coculture.

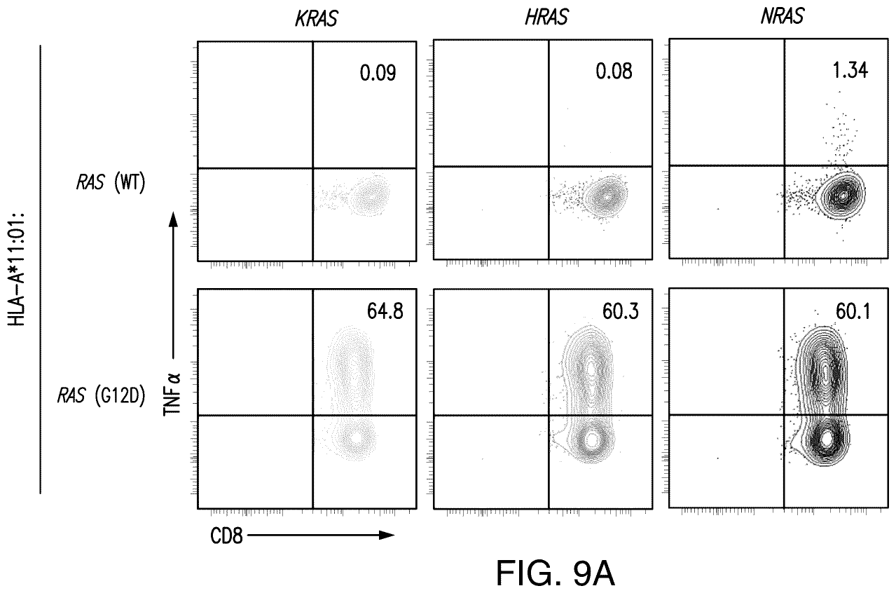

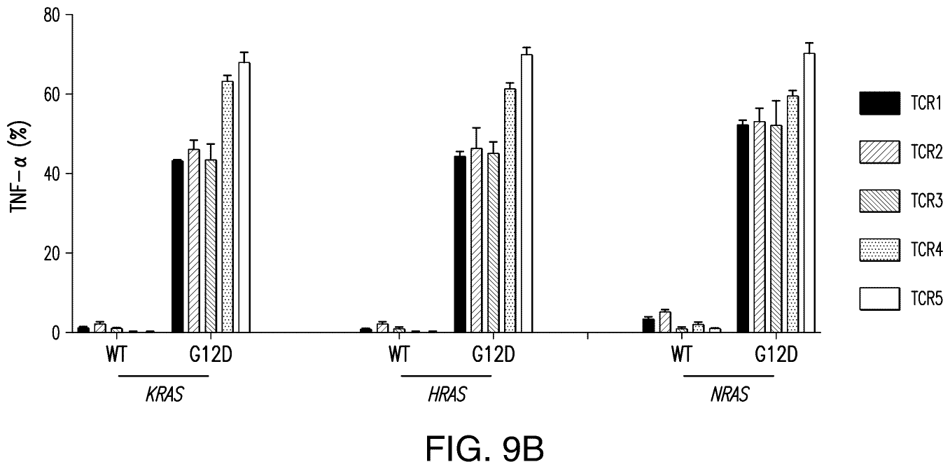

To test if individual library members could afford cross-protection against alternative mutant RAS proteins, TCR-transduced T cells were co-cultured with HLA-A*11:01+ targets co-expressing individual G12D RAS isoforms (KRAS, HRAS and NRAS). WT RAS isoforms were used as specificity controls. Intracellular TNF-α production was determined in CD8+ T cells expressing the transduced TCR. As shown in FIGS. 9A and 9B, cross-protective function of all five RAS(G12D)-specific TCRs was observed.

### Example 2

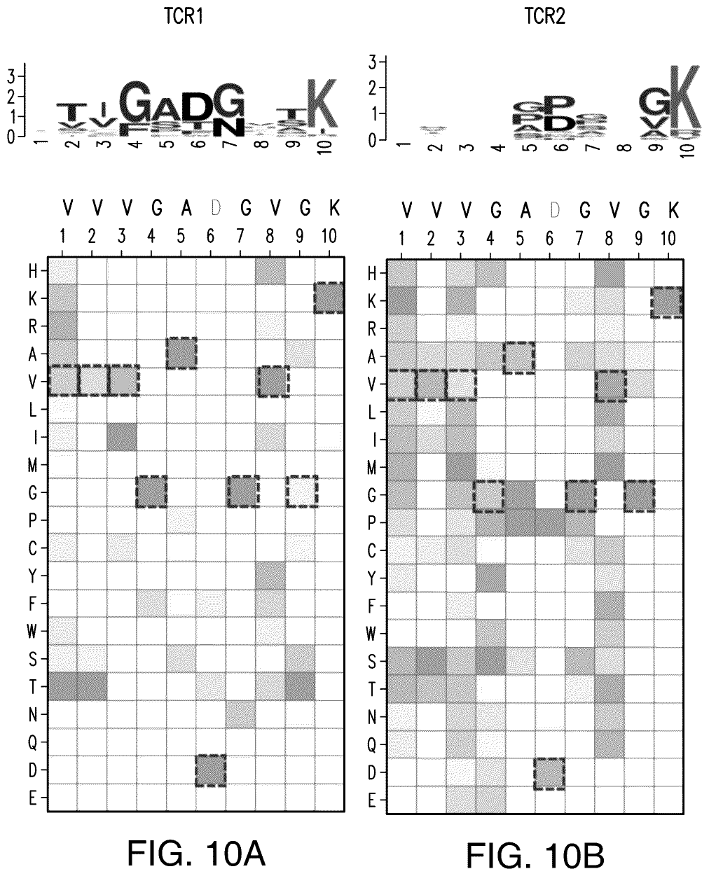

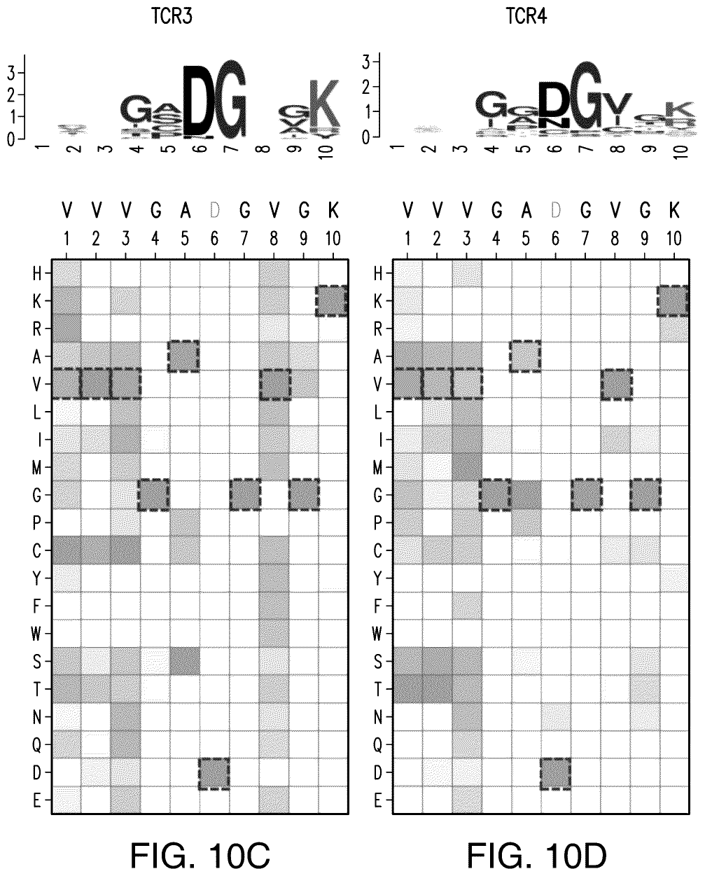

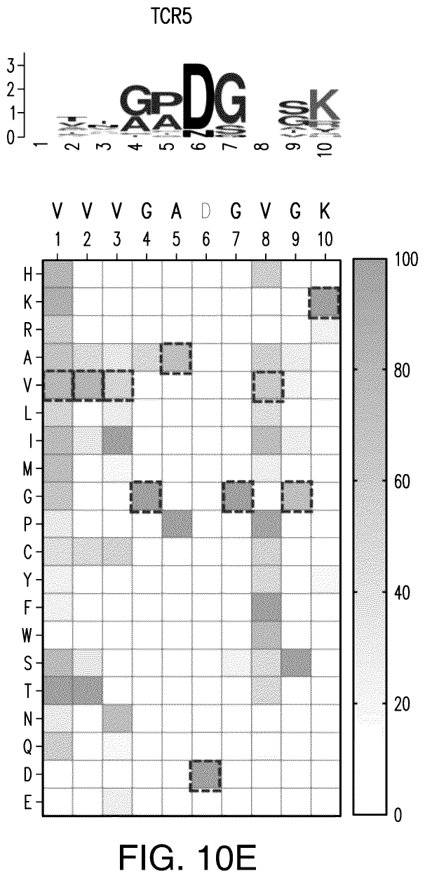

To identify TCR cross-reactivity profile, positional library scanning experiments were performed. A positional scanning library (PSL) was synthesized by substituting each amino acid (AA) in the index 10-mer RAS mutated peptide sequence set forth in SEQ ID NO: 2 (e.g., VVVGADGVGK) with every other amino acid. Target COS-7 cells were electroporated with mRNA encoding full-length human HLA*11:01 and incubated at 37 degrees overnight to allow HLA-A*11:01 protein expression. HLA*11:01⁺ target wells were then pulsed with an individual peptide (at a concentration of 1 μM) from the PSL; wild-type (WT) and mutated RAS peptides were included as functional controls. RAS TCR-T cells, expressing individual RAS TCRs 1-5, were added at an E:T ratio of 1:1 and incubated at 37 degrees for 24 h. An ELISA assay was performed on supernatants harvested from coculture wells to determine levels of IFN-γ production. Levels of IFN-γ production relative to the index amino acid at each position were calculated and plotted as shown in the heatmaps in FIGS. 10A-10E. As shown in FIGS. 10A-10E, TCR logo plots above each individual TCR heatmap show the relative influence of each amino acid at every position.

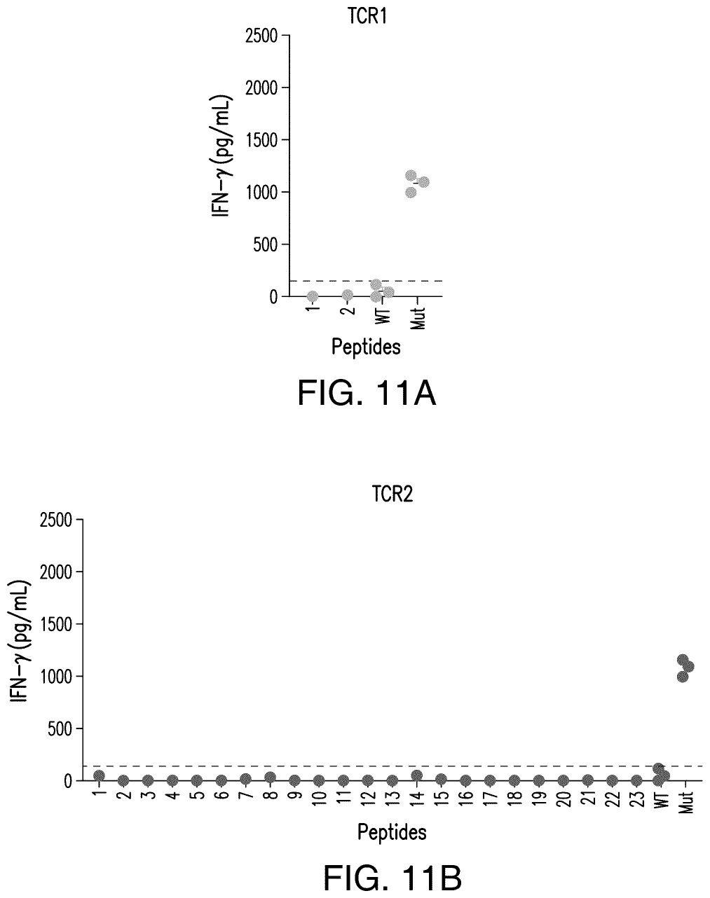

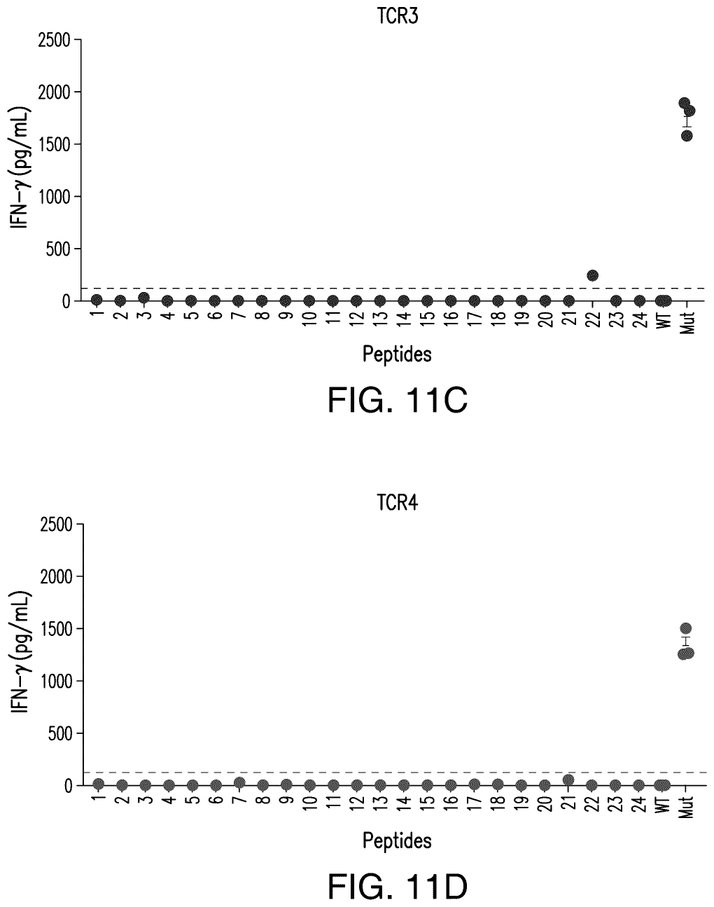

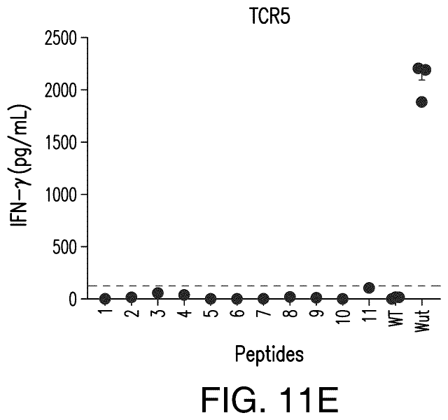

Next, studies were conducted to determine the cross-reactivity potential of RAS-specific TCRs. Individual TCR peptide motifs were scanned against the human proteome to identify potential cross-reactive sequences. Target COS-7 cells were electroporated with mRNA encoding full-length human HLA*11:01 and incubated at 37 degrees overnight to allow HLA-A*11:01 protein expression. HLA*11:01⁺ target wells. The cells were then pulsed with an individual peptide corresponding to its RAS-specific TCR (see Tables 7-11) at a concentration of 1 μM; finally, wild-type (WT) and mutated RAS peptides were included as functional controls. RAS TCR-T cells, expressing individual RAS TCRs 1-5, were added at an E:T ratio of 1:1 and incubated at 37 degrees for 24 h. Supernatants were harvested from coculture wells to perform an ELISA assay to determine levels of IFN-γ production. IFN-γ production is shown in FIGS. 11A-11E.

TABLE 7
List of identified peptides tested against
TCR1 to determine cross-reactive potential

| | | | | SEQ | Binding |
|---|---|---|---|---|---|
| | Peptide | Protein | | ID | Affinity |
| TCR | # | (Human) | Sequence | NO | (nM) |
| TCR1 | 1 | GEM | AVFGAGGVGK | 59 | 17.84 |
| | 2 | R-Ras | VVVGGGGVGK | 60 | 280.45 |

TABLE 8
List of identified peptides tested against
TCR2 to determine cross-reactive potential

| | | | | SEQ | Binding |
|---|---|---|---|---|---|
| | Peptide | Protein | | ID | Affinity |
| TCR | # | (Human) | Sequence | NO | (nM) |
| TCR2 | 1 | ABCF1 | STSPSDKVVK | 61 | 61.8 |
| | 2 | BMS1 | VVMGPPKVGK | 62 | 19 |
| | 3 | CD166 | NVFEAPTIVK | 63 | 34.17 |
| | 4 | CFA47 | MVFDSPTIGK | 64 | 9.63 |
| | 5 | CK049 | YSCPPPALVK | 65 | 92.39 |
| | 6 | CL056 | SSQSAPTTGK | 66 | 29.3 |
| | 7 | CPNS1 | AVMDSDTTGK | 67 | 17.85 |
| | 8 | CPNS2 | SVMDSDTTGK | 68 | 14.26 |
| | 9 | FLNA | VTIDGPSKVK | 69 | 110.77 |
| | 10 | GRIN1 | GTAGPPSAVK | 70 | 34.06 |
| | 11 | IGM | LTESGPALVK | 71 | 89.82 |
| | 12 | KZF4 | HSVSSPTVGK | 72 | 31.64 |
| | 13 | IL17F | KTLHGPAMVK | 73 | 14.61 |
| | 14 | K1671 | TTKSGPALGK | 74 | 43.36 |
| | 15 | KAD3 | VIMGAPGSGK | 75 | 61.62 |
| | 16 | LEGL | SVADSDAVVK | 76 | 37.46 |
| | 17 | RL7A | KVAPAPAVVK | 77 | 26.07 |
| | 18 | RSLAA | AVLGAPGVGK | 78 | 23.04 |
| | 19 | SALL1 | SATSPPGSVK | 79 | 57.42 |
| | 20 | SEZ6 | LSLEAPTVGK | 80 | 36.85 |
| | 21 | SHAN3 | TTVPSPASGK | 81 | 40.53 |
| | 22 | GEM | AVFGAGGVGK | 82 | 17.84 |
| | 23 | R-Ras | VVVGGGGVGK | 83 | 280.45 |

TABLE 9
List of identified peptides tested against
TCR3 to determine cross-reactive potential

| | Pep- | | | SEQ | Binding |
|---|---|---|---|---|---|
| | tide | Protein | | ID | Affinity |
| TCR | # | (Human) | Sequence | NO | (nM) |
| TCR3 | 1 | CHADL | RQCGADKVGK | 84 | 2054.06 |
| | 2 | DAZP1 | NNSGADEIGK | 85 | 10024.55 |
| | 3 | FMNL1 | QEAGADTPGK | 86 | 8376.76 |
| | 4 | GTR14 | QAHGADRSGK | 87 | 7606.17 |
| | 5 | GTR3 | QAHGADRSGK | 88 | 7606.17 |
| | 6 | MICU1 | YFFGADLKGK | 89 | 1261.65 |
| | 7 | ADA2C | GAGGADGQGA | 90 | 44077.09 |
| | 8 | CC88B | LRLGADGAGS | 91 | 42903.6 |
| | 9 | DYH8 | KVAGADGKGI | 92 | 35408.74 |
| | 10 | EFMT2 | MSSGADGGGG | 93 | 38441.82 |
| | 11 | FOXL2 | AGAGADGYGY | 94 | 11757.47 |
| | 12 | HMCN2 | IKQGADGSGT | 95 | 46453.35 |
| | 13 | MARF1 | LKLGADGSGP | 96 | 45513.05 |
| | 14 | MED13 | NNDGADGMGI | 97 | 39476.61 |
| | 15 | MYLK | GGVGADGGGS | 98 | 44273.06 |
| | 16 | NDF2 | TEQGADGAGR | 99 | 22110.67 |
| | 17 | PBX3 | GHEGADGDGR | 100 | 37763.65 |
| | 18 | PER1 | PLEGADGGGD | 101 | 48410.95 |
| | 19 | ZN646 | PEDGADGWGP | 102 | 47158.29 |
| | 20 | HIC1 | GVPGPDGKGK | 103 | 5447.86 |
| | 21 | TBL3 | LSSGSDGLVK | 104 | 386.01 |
| | 22 | TUTLA | ISQGADGRGK | 105 | 2512.76 |
| | 23 | GEM | AVFGAGGVGK | 106 | 17.84 |
| | 24 | R-Ras | VVVGGGGVGK | 107 | 280.45 |

TABLE 10
List of identified peptides tested against
TCR4 to determine cross-reactive potential

| | Pep- | | | SEQ | Binding |
|---|---|---|---|---|---|
| | tide | Protein | | ID | Affinity |
| TCR | # | (Human) | Sequence | NO | (nM) |
| TCR4 | 1 | BY55 | RDPGIDGVGE | 108 | 41650.79 |
| | 2 | CO6A3 | GDDGRDGVGS | 109 | 45516.02 |
| | 3 | CO8A2 | GPPGVDGVGV | 110 | 42319.92 |
| | 4 | COBA1 | GPAGQDGVGG | 111 | 44849.22 |
| | 5 | COIA1 | GDPGKDGVGQ | 112 | 44614.95 |
| | 6 | EFGM | EVKGKDGVGA | 113 | 42587.71 |
| | 7 | MEIS1 | HYGGMDGVGI | 114 | 38054.46 |
| | 8 | MEIS2 | HYGGMDGVGV | 115 | 38131.13 |
| | 9 | PUR2 | LASGTDGVGT | 116 | 40367.23 |
| | 10 | USH1G | EDGGLDGVGA | 117 | 44344.5 |
| | 11 | ABCF1 | CIVGPNGVGK | 118 | 241.96 |
| | 12 | AGRIN | PVCGSDGVTY | 119 | 20982.35 |
| | 13 | CGAT2 | RNVGANGIGY | 120 | 3473.24 |
| | 14 | DNHD1 | TVLGPNGVGK | 121 | 31.32 |
| | 15 | IBP7 | PVCGSDGTTY | 122 | 25802.72 |
| | 16 | NLRP9 | VLEGPDGIGK | 123 | 1072.06 |
| | 17 | PRA19 | KLFISDGCGY | 124 | 2620.21 |
| | 18 | SMC5 | MIVGANGTGK | 125 | 432.29 |
| | 19 | SO3A1 | PVCGADGITY | 126 | 19587.46 |
| | 20 | SO5A1 | PVCGSDGITY | 127 | 19288.81 |
| | 21 | SOCS7 | GSGGGDGTGK | 128 | 2094.6 |
| | 22 | SUCB2 | CAIIANGITK | 129 | 167.25 |
| | 23 | GEM | AVFGAGGVGK | 130 | 17.84 |
| | 24 | R-Ras | VVVGGGGVGK | 131 | 280.45 |

TABLE 11
List of identified peptides tested against
TCR5 to determine cross-reactive potential

| | | | | SEQ | Binding |
|---|---|---|---|---|---|
| | Peptide | Protein | | ID | Affinity |
| TCR | # | (Human) | Sequence | NO | (nM) |
| TCR5 | 1 | FRPD4 | KSKLADGEGK | 132 | 947.24 |
| | 2 | TAF6 | GATTADGKGK | 133 | 5042.79 |
| | 3 | TUTLA | ISQGADGRGK | 134 | 2512.76 |
| | 4 | DCAF7 | ASVGADGSVR | 135 | 2514.45 |
| | 5 | ELP2 | VSAAADSAVR | 136 | 1607.6 |
| | 6 | FRPD1 | KVAAADGPAR | 137 | 738.49 |
| | 7 | INO80 | SSLAPDSLVR | 138 | 159.12 |
| | 8 | KI26A | PVAGPDGLSK | 139 | 1253.22 |
| | 9 | PRP4 | ASCAADGSVK | 140 | 173.07 |
| | 10 | GEM | AVFGAGGVGK | 141 | 17.84 |
| | 11 | R-Ras | VVVGGGGVGK | 142 | 280.45 |

As shown in FIGS. 11A-11E, each TCR exhibited a functional response when incubated with an HLA-A*11:01⁺ cell presenting the mutated RAS peptide (e.g., the mutated RAS peptide consisting of the amino acid sequence set forth in SEQ ID NO: 2) but not the corresponding wild type sequence (VVVGAGGVGK). TCRs 1, 2, 4, and 5 did not exhibit reactivity to alternative human peptide sequences which possess a recognition motif elucidated in FIGS. 10A-10E. TCR 3 exhibited low level reactivity to a single alternative peptide (TCR 3, peptide 22; table 9) when pulsed at non physiologic concentrations onto an HLA-A*11:01⁺ target cell population. Thus, these data demonstrate that the presently disclosed TCRs can specifically bind to the mutated RAS peptide and induce specific T cell activation with negligible reactivity to alternative peptide species.

Embodiments of the presently disclosed subject matter

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A T cell receptor (TCR) that binds to a RAS peptide comprising a G12 mutation, wherein TCR comprises an α chain comprising an α chain variable region and α chain constant region, and a β chain comprising a β chain variable region and a β chain constant region, wherein
a) the α chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and the β chain variable region comprises a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
b) the α chain variable region comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and the β chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19;
c) the α chain variable region comprises a CDR1 comprising the amino acid sequence set forth in SEQ ID NO: 24, a CDR2 comprising the amino acid sequence set forth in SEQ ID NO: 15, and a CDR3 comprising the amino acid sequence set forth in SEQ ID NO: 25; and the β chain variable region comprises a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28;
d) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; or
e) the α chain variable region comprises a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and the β chain variable region comprises a CDR1 comprising the amino acid sequence set forth in SEQ ID NO: 58, a CDR2 comprising the amino acid sequence set forth in SEQ ID NO: 46, and a CDR3 comprising the amino acid sequence set forth in SEQ ID NO: 47.

2. The TCR of claim 1, wherein the RAS peptide comprises or consists of the amino acid sequence set forth in SEQ ID NO: 1 or SEQ ID NO: 2.

3. The TCR of claim 1, wherein the RAS peptide is associated with an HLA-A.

4. The TCR of claim 3, wherein the HLA-A is an HLA-A*03 superfamily member.

5. The TCR of claim 4, wherein the HLA-A*03 superfamily member is selected from the group consisting of HLA-A*03, HLA-A*11, HLA-A*31, HLA-A*33, HLA-A*66, HLA-A*68 and HLA-A*74.

6. The TCR of claim 5, wherein the HLA-A*03 superfamily member is HLA-A*11.

7. The TCR of claim 1, wherein the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the β chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

8. The TCR of claim 1, wherein:
a) the α chain variable region comprises an amino acid sequence that is at least about 80% identical to the amino acid sequence set forth in SEQ ID NO: 10, and the β chain variable region comprises an amino acid sequence that is at least about 80% identical to the amino acid sequence set forth in SEQ ID NO: 11;
b) the α chain variable region comprises an amino acid sequence that is at least about 80% identical to the amino acid sequence set forth in SEQ ID NO: 20, and the β chain variable region comprises an amino acid sequence that is at least about 80% identical to the amino acid sequence set forth in SEQ ID NO: 21;
c) the α chain variable region comprises an amino acid sequence that is at least about 80% identical to the amino acid sequence set forth in SEQ ID NO: 29, and the β chain variable region comprises an amino acid sequence that is at least about 80% identical to the amino acid sequence set forth in SEQ ID NO: 30;
d) the α chain variable region comprises an amino acid sequence that is at least about 80% identical to the amino acid sequence set forth in SEQ ID NO: 39, and the β chain variable region comprises an amino acid sequence that is at least about 80% identical to the amino acid sequence set forth in SEQ ID NO: 40; or
e) the α chain variable region comprises an amino acid sequence that is at least about 80% identical to the amino acid sequence set forth in SEQ ID NO: 48, and the β chain variable region comprises an amino acid sequence that is at least about 80% identical to the amino acid sequence set forth in SEQ ID NO: 49.

9. The TCR of claim 8, wherein:
a) the α chain variable region comprises the amino acid sequence set forth in SEQ ID NO: 10, and the β chain variable region comprises the amino acid sequence set forth in SEQ ID NO: 11;
b) the α chain variable region comprises the amino acid sequence set forth in SEQ ID NO: 20, and the β chain variable region comprises the amino acid sequence set forth in SEQ ID NO: 21;
c) the α chain variable region comprises the amino acid sequence set forth in SEQ ID NO: 29, and the β chain variable region comprises the amino acid sequence set forth in SEQ ID NO: 30;
d) the α chain variable region comprises the amino acid sequence set forth in SEQ ID NO: 39, and the β chain variable region comprises the amino acid sequence set forth in SEQ ID NO: 40; or
e) the α chain variable region comprises the amino acid sequence set forth in SEQ ID NO: 48, and the β chain variable region comprises the amino acid sequence set forth in SEQ ID NO: 49.

10. The TCR of claim 1, wherein:
a) the α chain comprises the amino acid sequence set forth in SEQ ID NO: 12, and the β chain comprises the amino acid sequence set forth in SEQ ID NO: 13;
b) the α chain comprises the amino acid sequence set forth in SEQ ID NO: 22, and the β chain comprises the amino acid sequence set forth in SEQ ID NO: 23;
c) the α chain comprises the amino acid sequence set forth in SEQ ID NO: 31, and the β chain comprises the amino acid sequence set forth in SEQ ID NO: 32;
d) the α chain comprises the amino acid sequence set forth in SEQ ID NO: 41, and the β chain comprising the amino acid sequence set forth in SEQ ID NO: 42; or
e) the α chain comprises the amino acid sequence set forth in SEQ ID NO: 50, and the β chain comprises the amino acid sequence set forth in SEQ ID NO: 51.

11. The TCR of claim 1, wherein the TCR is recombinantly expressed, and/or expressed from a vector.

12. The TCR of claim 1, wherein the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 53 or SEQ ID NO: 54; and/or the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 55, SEQ ID NO: 56, or SEQ ID NO: 57.

13. The TCR of claim 12, wherein the α chain constant region comprises the amino acid sequence set forth in SEQ ID NO: 53 or SEQ ID NO: 54; and/or the β chain constant region comprises the amino acid sequence set forth in SEQ ID NO: 55, SEQ ID NO: 56, or SEQ ID NO: 57.

14. A nucleic acid encoding the T cell receptor (TCR) of claim 1.

15. An immunoresponsive cell comprising the TCR of claim 1.

16. The immunoresponsive cell of claim 15, wherein the TCR is constitutively expressed on the surface of the cell.

17. The immunoresponsive cell of claim 15, wherein the cell is selected from the group consisting of a T cell, and a pluripotent stem cell from which a lymphoid cell may be differentiated.

18. The immunoresponsive cell of claim 17, wherein the cell is a T cell.

19. The immunoresponsive cell of claim 18, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

20. The cell of claim 15, wherein the TCR or the nucleic acid is integrated at a locus within the genome of the cell.

21. The cell of claim 20, wherein the locus is selected from a TRAC locus, a TRBC locus, a TRDC locus, and a TRGC locus.

22. A pharmaceutical composition comprising the cell of claim 15 and a pharmaceutically acceptable carrier.

23. A vector comprising the nucleic acid of claim 14.

24. A method for producing a cell that binds to a RAS peptide that comprises a G12 mutation, comprising introducing into the cell the nucleic acid of claim 14.

25. A method of treating and/or preventing a tumor associated with RAS in a subject, comprising administering to the subject the cell of claim 15.

26. The method of claim 25, wherein the tumor is selected from the group consisting of pancreatic cancer, breast cancer, endometrial cancer, cervical cancer, anal cancer, bladder cancer, colorectal cancer, cholangiocarcinoma/bile duct cancer, lung cancer, ovarian cancer, esophageal cancer, gastric cancer, head and neck squamous cell carcinoma, nonmelanoma skin cancer, salivary gland cancer, melanoma, and multiple myeloma.

27. The method of claim 25, wherein the subject comprises an HLA-A.

28. The method of claim 27, wherein the HLA-A is an HLA-A*03 superfamily member.

29. The method of claim 28, wherein the HLA-A*03 superfamily member is selected from the group consisting of HLA-A*03, HLA-A*11, HLA-A*31, HLA-A*33, HLA-A*66, HLA-A*68 and HLA-A*74.

30. A T cell receptor (TCR) that binds to a RAS peptide, wherein the RAS peptide comprises comprising a G12 mutation, wherein TCR comprises an α chain comprising an α chain variable region and α chain constant region, and a β chain comprising a β chain variable region and a β chain constant region, wherein
a) the α chain variable region comprises a CDR1, a CDR2, and a CDR3 of the α chain variable region comprising the amino acid sequence set forth in SEQ ID NO: 10, and the β chain variable region comprises a CDR1, a CDR2, and a CDR3 of the β chain variable region comprising the amino acid sequence set forth in SEQ ID NO: 11;
b) the α chain variable region comprises a CDR1, a CDR2, and a CDR3 of the α chain variable region comprising the amino acid sequence set forth in SEQ ID NO: 20, and the β chain variable region comprises a CDR1, a CDR2, and a CDR3 of the β chain variable region comprising the amino acid sequence set forth in SEQ ID NO: 21;
c) the α chain variable region comprises a CDR1, a CDR2, and a CDR3 of the α chain variable region comprising the amino acid sequence set forth in SEQ ID NO: 29, and the β chain variable region comprises a CDR1, a CDR2, and a CDR3 of the β chain variable region comprising the amino acid sequence set forth in SEQ ID NO: 30;
d) the α chain variable region comprises a CDR1, a CDR2, and a CDR3 of the α chain variable region comprising the amino acid sequence set forth in SEQ ID NO: 39, and the β chain variable region comprises a CDR1, a CDR2, and a CDR3 of the β chain variable region comprising the amino acid sequence set forth in SEQ ID NO: 40; or
e) the α chain variable region comprises a CDR1, a CDR2, and a CDR3 of the α chain variable region comprising the amino acid sequence set forth in SEQ ID NO: 48, and the β chain variable region comprises a CDR1, a CDR2, and a CDR3 of the β chain variable region comprising the amino acid sequence set forth in SEQ ID NO: 49.
